(19) United States
(12) Patent Application Publication
(10) Pub. No.: US 2026/0265776 A1
(43) Pub. Date: Sep. 10, 2026

(54) ENGINEERED RECOMBINANT RETROVIRUSES

(71) Applicant: Kelonia Therapeutics, Inc., Boston, MA (US)

(72) Inventors: Kevin M. Friedman, Boston, MA (US); Michael J. McDonald, Boston, MA (US)

(21) Appl. No.: 19/166,370

(22) PCT Filed: Mar. 15, 2024
(86) PCT No.: PCT/US2024/020087

§ 371 Date: Sep. 17, 2025

Related U.S. Application Data
(60) Provisional application No. 63/453,010, filed on Mar. 17, 2023.
(60) Provisional application No. 63/532,085, filed on Aug. 11, 2023.

(51) Int. Cl. C12N 15/86; C07K 14/005; C07K 16/28

(52) U.S. Cl. CPC C12N 15/86; C07K 14/005; C07K 16/2809; C12N 2740/10043; C12N 2740/10052; C12N 2760/18222; C12N 2760/18422; C12N 2760/20222

(57) ABSTRACT

The present disclosure provides recombinant retroviruses engineered to transduce immune effector cells in vivo.

### CROSS REFERENCE TO RELATED APPLICATIONS

This application claims the benefit under 35 U.S. C. § 119(e) of U.S. Provisional Application No. 63/453,010, filed Mar. 17, 2023, and U.S. Provisional Application No. 63/532,085, filed Aug. 11, 2023, and each of which is incorporated by reference herein in its entirety.

### STATEMENT REGARDING SEQUENCE LISTING

The Sequence Listing associated with this application is provided in .xml format in lieu of a paper copy and is hereby incorporated by reference into the specification. The name of the .xml file containing the Sequence Listing is KELO-006-WO1_ST26.xml. The .xml file is 143 KB, was created on Mar. 14, 2024, and is being submitted electronically via Patent Center, concurrent with the filing of the specification.

### TECHNICAL FIELD

The present disclosure relates to engineered recombinant retroviruses, compositions, and associated methods. More particularly, the disclosure relates to recombinant retroviruses engineered to transduce immune effector cells in vivo.

### DESCRIPTION OF THE RELATED ART

Various vectors suitable for gene therapy are constrained by size limits of the polynucleotides to be delivered. Expensive manufacturing strategies also heavily contribute to the obstacles preventing widespread adoption of gene therapies. And for systems with larger limits, in vivo delivery has been challenging.

### BRIEF SUMMARY

The present disclosure generally relates, in part, to recombinant retroviruses for in vivo delivery of engineered antigen receptors to immune effector cells, compositions, methods of making the recombinant retroviruses, and methods of using the recombinant retroviruses to treat a subject in need thereof.

In various embodiments, the disclosure contemplates a recombinant retrovirus comprising (a) a viral envelope comprising (i) one or more mutated viral envelope glycoproteins that retain fusogenic activity and lack cognate receptor binding activity and (ii) a non-viral membrane-bound tropism polypeptide, and (b) a recombinant retroviral vector comprising a polynucleotide encoding a promoter operably linked to a polynucleotide encoding one or more engineered antigen receptors; wherein the retroviral vector is engineered or derived from a retrovirus selected from the group consisting of: an alpharetrovirus genome, a betaretrovirus genome, a gammaretrovirus genome, a deltaretrovirus genome, an epsilonretrovirus genome, a simiispumavirus genome, a bovispumavirus genome, an equispumavirus genome, a felispumavirus genome, and a prosimiispumavirus genome.

In particular embodiments, the retrovirus comprises a gag protein and a pol protein of an alpharetrovirus, a betaretrovirus, a gammaretrovirus, a deltaretrovirus, an epsilonretrovirus, a simiispumavirus, a bovispumavirus, an equispumavirus, a felispumavirus, or a prosimiispumavirus; and a 5′ LTR and a 3′ LTR isolated, obtained, or derived from a retrovirus genome selected from the group consisting of: an alpharetrovirus genome, a betaretrovirus genome, a gammaretrovirus genome, a deltaretrovirus genome, an epsilonretrovirus genome, a simiispumavirus genome, a bovispumavirus genome, an equispumavirus genome, a felispumavirus genome, and a prosimiispumavirus genome.

In certain embodiments, the one or more mutated viral envelope glycoproteins comprise a vesiculovirus envelope glycoprotein, one or more morbillivirus envelope glycoproteins or one or more henipavirus envelope glycoproteins.

In some embodiments, the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In further embodiments, the vesiculovirus envelope glycoprotein is a vesiculovirus G protein.

In additional embodiments, the vesiculovirus G protein is a COCV G glycoprotein (COCV-G) or a VSIV G glycoprotein (VSIV-G).

In particular embodiments, the VSIV-G envelope protein comprises one or more of: (a) one or more amino acid substitutions at H8, N9, Q10, K47, K50, A51, S183, S179, N180, I182, M184, Y209, T214, I347, T350, T352, E353, and R354; (b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; (c) amino acid substitutions at K47 and/or R354; or (d) a deletion of residues 1-8.

In particular embodiments, the VSIV-G envelope protein comprises one or more amino acid substitutions at H8, K47, Y209, and R354.

In certain embodiments, the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In particular embodiments, the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

In further embodiments, the vesiculovirus G protein is COCV-G.

In particular embodiments, the COCV-G envelope protein comprises one or more amino acid substitutions at K47 and R354.

In additional embodiments, the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In particular embodiments, the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=A and X₂=Q.

In particular embodiments, the one or more morbillivirus envelope glycoproteins are measles virus F (MV-F) and measles virus H (MV-H).

In certain embodiments, the MV-H protein comprises one or more amino acid substitutions at Y481, R533, S548, and F549.

In particular embodiments, the MV-H protein comprises one or more amino acid substitutions selected from the group consisting of: Y481A, R533A, S548L, and F549S.

In particular embodiments, the one or more henipavirus envelope glycoproteins are nipah virus F (NiV-F) and nipah virus G (NiV-G).

In additional embodiments, the NiV-G protein comprises one or more amino acid substitutions at E501, W504, Q530, and E533.

In further embodiments, the NiV-G protein comprises one or more amino acid substitutions at E501A, W504A, Q530A, and E533A.

In particular embodiments, the non-viral membrane-bound tropism polypeptide comprises an extracellular antigen targeting domain, a spacer domain, and a transmembrane domain.

In particular embodiments, the extracellular antigen targeting domain binds an antigen expressed on an immune effector cell.

In certain embodiments, the extracellular antigen targeting domain binds an antigen expressed on an immune effector cell selected from the group consisting of: the alpha, beta, gamma, or delta chain of the T cell receptor, CD2, CD3δ, CD3ε, CD3γ, CD4, CD5, CD7, CD8α, and CD8β.

In further embodiments, the extracellular antigen targeting domain comprises an antibody or antigen binding fragment thereof that binds an antigen expressed on the immune effector cell.

In additional embodiments, the extracellular antigen targeting domain comprises an anti-CD3 antibody or antigen binding fragment selected from the group consisting of OKT3, UCHT1, YTH12.5, TR66, and humanized variants thereof, e.g., teplizumab, and antibodies and antigen binding fragments that have at least 80%, at least 85%, at least 90%, at least 95%, at least 99%, or 100% identity thereto.

In particular embodiments, the viral envelope further comprises one or more secondary tropism polypeptides.

In certain embodiments, the one or more secondary tropism polypeptides comprise one or more of CD80, CD86, CD137L, OX40L, and ICOSL.

In particular embodiments, the promoter is an EF1α promoter, a cytomegalovirus (CMV) promoter, a CAG promoter, an SV40 promoter, an SV40/CD43 promoter, a murine stem cell virus (MSCV) promoter, a phosphoglycerate kinase (PGK) promoter, or an MNDU3 promoter.

In some embodiments, the engineered antigen receptor is a chimeric antigen receptor (CAR), a T cell receptor (TCR), or a dimerization activated receptor initiation complex (DARIC).

In some embodiments, the engineered antigen receptor comprises an extracellular antigen binding domain that binds to alpha folate receptor (FRα), αvβ6 integrin, B cell maturation antigen (BCMA), B7-H3 (CD276), B7-H6, carbonic anhydrase IX (CAIX), CD16, CD 19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant IP (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), G Protein-Coupled Receptor Class C Group 5 Member D (GPCR5D), EGFR family including ErbB2 (HER2), IL-10Rα, IL-13Rα2, Kappa, cancer/testis antigen 2 (LAGE-1A), Lambda, Lewis-Y (LeY), LI cell adhesion molecule (L1-CAM), melanoma antigen gene (MAGE)-A1, MAGE-A3, MAGE-A4, MAGE-A6, MAGE A10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL 16-binding protein (ULBP) 1, ULBP2, ULBP3, ULBP4, ULBP5, ULBP6, vascular endothelial growth factor receptor 2 (VEGFR2), or Wilms tumor 1 (WT-1).

In further embodiments, the engineered antigen receptor is a CAR.

In particular embodiments, the CAR comprises an extracellular antigen binding domain comprising an antibody or antigen binding fragment thereof.

In additional embodiments, the antibody or antigen binding fragment thereof is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), a single-domain antibody (sdAb, a camelid VHH, Nanobody) and a centyrin.

In certain embodiments, the antibody or antigen binding fragment thereof comprises one or more scFv and/or one or more VHHs.

In additional embodiments, the CAR comprises a hinge domain, optionally a hinge domain selected from the group consisting of an IgG4 hinge, a CD8α hinge, and a CD28 hinge.

In particular embodiments, the CAR comprises a transmembrane domain isolated, obtained, or derived from a polypeptide selected from the group consisting of the alpha, beta, gamma, or delta chain of the T cell receptor, CD2, CD3, CD4, CD8α, and CD28.

In some embodiments, the CAR comprises one or more intracellular primary signaling domains isolated, obtained, or derived from a polypeptide selected from the group consisting of: FcRγ, FcRβ, CD3ζ, CD79a, CD79b, and CD66d.

In certain embodiments, the CAR comprises one or more intracellular costimulatory signaling domains isolated, obtained, or derived from a polypeptide selected from the group consisting of: CD2, CD7, CD11a, CD27, CD28, CD30, CD40, CD134 (OX40), CD137 (4-1BB), CD278 (ICOS), and TNF receptor superfamily member 14 (TNFRS14).

In particular embodiments, the CAR comprises an amino acid sequence set forth in any one of SEQ ID NOs: 93 to 97.

In some embodiments, the retroviral vector further comprises one or more polynucleotides encoding a P2A self-cleaving peptide, a T2A self-cleaving peptide, or an E2A self-cleaving peptide.

In particular embodiments, the retroviral vector further comprises a polynucleotide encoding a prodrug activator enzyme.

In particular embodiments, the prodrug activator enzyme is cytosine deaminase or thymidine kinase.

In various embodiments, the disclosure contemplates a method of manufacturing a recombinant retrovirus contemplated herein, comprising transfecting a packaging cell line that expresses the one or more mutated viral envelope glycoproteins that retain fusogenic activity and lack cognate receptor binding activity, the non-viral membrane-bound tropism polypeptide, optionally the secondary tropism polypeptide, and a gag and pol contemplated herein, with a plasmid encoding the retroviral vector; and culturing the packaging cell to manufacture the recombinant retrovirus.

In various embodiments, the disclosure contemplates a method of manufacturing a recombinant retrovirus contemplated herein, comprising transfecting a packaging cell line that expresses a gag and pol contemplated herein with one or more plasmids encoding the one or more mutated viral envelope glycoproteins that retain fusogenic activity and lack cognate receptor binding activity, the non-viral membrane-bound tropism polypeptide, and optionally the secondary tropism polypeptide; and culturing the packaging cell to manufacture the recombinant retrovirus.

In various embodiments, the disclosure contemplates a composition comprising a recombinant retrovirus contemplated herein.

In various embodiments, the disclosure contemplates a pharmaceutical composition comprising a recombinant retrovirus contemplated herein and a pharmaceutically acceptable carrier.

In various embodiments, the disclosure contemplates a method of treating a subject in need thereof comprising administering to the subject an effective amount of a recombinant retrovirus contemplated herein.

In particular embodiments, the amount is effective to transduce an immune effector cell in the subject.

Further aspects and embodiments of the disclosure are provided by the following Detailed Description.

### BRIEF DESCRIPTION OF THE SEQUENCE IDENTIFIERS

SEQ ID NOs: 1-10 set forth amino acid sequences of fusogens.

SEQ ID NOs: 11-12 set forth amino acid sequences of anti-CD3 antibodies.

SEQ ID NOs: 13-92 set forth amino acid sequences of fusogens.

SEQ ID NOs: 93-97 set forth amino acid sequences of chimeric antigen receptors.

SEQ ID NOs: 98-111 set forth amino acid sequences of linker polypeptides.

In the foregoing sequences, X, if present, refers to any amino acid, a specified group of amino acids or the absence of an amino acid.

Throughout the disclosure, the amino acid position(s) of a fusogen is with reference to the fusogen lacking a signal sequence (i.e., the amino acid sequence after the signal peptide has been cleaved).

### DETAILED DESCRIPTION

### A. Overview

The present disclosure generally relates to, in part, recombinant retroviruses and retroviral vectors that lack lentivirus and lentiviral vector components, polypeptides, and polynucleotide sequences. More particularly, the present disclosure relates, in part, to recombinant retroviruses for in vivo delivery of engineered antigen receptors to immune effector cells, compositions, methods of making the recombinant retroviruses, and methods of using the recombinant retroviruses to treat a subject in need thereof.

The recombinant retrovirus comprises a viral envelope engineered to transduce target cells in vivo. The viral envelope comprises one or more mutated viral envelope glycoproteins that retain fusogenic activity and lack cognate receptor binding activity (i.e., modified to retain fusogenic activity and decrease, reduce, substantially ablate, ablate, abolish or eliminate cell binding or attachment activity) and a non-viral membrane-bound tropism polypeptide, and optionally a secondary tropism polypeptide.

The recombinant retrovirus comprises gag and pol from a non-lentiviral, retrovirus; a non-lentiviral, retroviral vector comprising a polynucleotide encoding a promoter operably linked to a promoter encoding one or more engineered antigen receptors to be expressed in the transduced target cell; wherein the retroviral vector is engineered or derived from a retrovirus selected from the group consisting of: an alpharetrovirus genome, a betaretrovirus genome, a gammaretrovirus genome, a deltaretrovirus genome, an epsilonretrovirus genome, a simiispumavirus genome, a bovispumavirus genome, an equispumavirus genome, a felispumavirus genome, and a prosimiispumavirus genome.

The present disclosure further contemplates compositions and pharmaceutical compositions comprising non-lentiviral recombinant retroviruses contemplated herein.

The present disclosure further contemplates a method of manufacturing recombinant non-lentiviral retroviruses contemplated herein and packaging cell lines for manufacturing the same.

The present disclosure further contemplates a method of treating a subject in need thereof comprising administering to the subject an effective amount of a recombinant non-lentiviral retrovirus contemplated herein.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 4th ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience (2002); Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid the Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells and Enzymes (IRL Press, 1986); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods in Cell and Molecular Biology (Mayer and Walker, eds., Academic Press, London, 1987); Handbook of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Additional definitions are set forth throughout this disclosure.

### C. Retroviruses

The present disclosure contemplates recombinant retroviruses that comprise proteins and nucleic acids derived from non-lentiviral sources. Recombinant retroviruses contemplated herein comprise a retroviral vector derived from non-lentiviral sources. The retroviral vector is based on a retrovirus genome that has been engineered to remove viral accessory proteins but leave elements intact for packaging, reverse transcription and integration. In particular embodiments, a recombinant virus contains two copies of a vector, a genomic RNA comprising backbone sequences derived from a retrovirus.

In various embodiments, a recombinant retrovirus comprises a recombinant retroviral vector engineered or derived from a retrovirus genome selected from the group consisting of: an alpharetrovirus genome, a betaretrovirus genome, a gammaretrovirus genome, a deltaretrovirus genome, or a spumavirus genome (e.g., an epsilonretrovirus genome, a simiispumavirus genome, a bovispumavirus genome, an equispumavirus genome, a felispumavirus genome, and a prosimiispumavirus genome).

In particular embodiments, a recombinant retrovirus comprises a gag protein and a pol protein of an alpharetrovirus, a betaretrovirus, a gammaretrovirus, a deltaretrovirus, an epsilonretrovirus, or a spumavirus.

In particular embodiments, a recombinant retrovirus comprises a 5′ LTR and a 3′ LTR each isolated, obtained, or derived from a retrovirus genome selected from the group consisting of: an alpharetrovirus genome, a betaretrovirus genome, a gammaretrovirus genome, a deltaretrovirus genome, an epsilonretrovirus genome, and a spumavirus genome.

Illustrative examples of alpharetroviruses include but are not limited to avian leukosis virus, avian carcinoma Mill Hill virus 2, avian myeloblastosis virus, avian myelocytomatosis virus 29, avian sarcoma virus CT10, fujinami sarcoma virus, rous sarcoma virus, UR2 sarcoma virus and Y73 sarcoma virus.

Illustrative examples of betaretroviruses include but are not limited to mouse mammary tumor virus, Jaagsiekte sheep retrovirus, langur virus, Mason-Pfizer monkey virus, and squirrel monkey retrovirus (SMRV).

Illustrative examples of deltaretroviruses include but are not limited to bovine leukemia virus, primate T-lymphotropic virus 1, primate T-lymphotropic virus 2, primate T-lymphotropic virus 3, and primate T-lymphotropic virus 4.

Illustrative examples of epsilonretroviruses include but are not limited to walleye dermal sarcoma virus, walleye epidermal hyperplasia virus 1, and walleye epidermal hyperplasia virus 2.

Illustrative examples of gammaretrovirus include but are not limited to baboon endogenous virus (BaEV), chick syncytial virus, feline endogenous virus (e.g., RD 114), feline leukemia virus (FeLV), Finkel-Biskis-Jinkins murine sarcoma virus, Gardner-Arnstein feline sarcoma virus, gibbon ape leukemia virus (GALV), guinea pig type-C oncovirus, Hardy-Zuckerman feline sarcoma virus, Harvey murine sarcoma virus, Kirsten murine sarcoma virus, koala retrovirus, murine leukemia virus (MLV), Moloney murine leukemia virus (MoMLV), Moloney murine sarcoma virus, porcine endogenous virus (PERV), Porcine type-C oncovirus, reticuloendotheliosis virus (REV), Snyder-Theilen feline sarcoma virus, Trager duck spleen necrosis virus, viper retrovirus, xenotropic murine leukemia virus-related virus (XMRV), and woolly monkey sarcoma virus.

Illustrative examples of spumaviruses include but are not limited to simian foamy virus, bovine foamy virus, equine foamy virus, feline foamy virus, human foamy virus (HFV), and brown greater galago prosimian foamy virus.

In particular embodiments, a recombinant retrovirus comprises a recombinant retroviral vector engineered or derived from a retrovirus genome selected from the group consisting of: SMRV, BaEV, RD114, FeLV, GALV, MLV, MoMLV, PERV, REV, XMRV, and HFV; gag and pol from an alpharetrovirus, a betaretrovirus, a gammaretrovirus, a deltaretrovirus, an epsilonretrovirus, or a spumavirus; and a 5′ LTR and a 3′ LTR each isolated, obtained, or derived from a retrovirus genome selected from the group consisting of: an alpharetrovirus genome, a betaretrovirus genome, a gammaretrovirus genome, a deltaretrovirus genome, an epsilonretrovirus genome, and a spumavirus genome.

### D. Viral Envelope

In particular embodiments, a recombinant comprises a recombinant retroviral vector engineered or derived from a retrovirus genome selected from the group consisting of: SMRV, BaEV, RD114, FeLV, GALV, MLV, MoMLV, PERV, REV, XMRV, and HFV; gag and pol from an alpharetrovirus, a betaretrovirus, a gammaretrovirus, a deltaretrovirus, an epsilonretrovirus, or a spumavirus; and a 5′ LTR and a 3′ LTR each isolated, obtained, or derived from a retrovirus genome selected from the group consisting of: an alpharetrovirus genome, a betaretrovirus genome, a gammaretrovirus genome, a deltaretrovirus genome, an epsilonretrovirus genome, and a spumavirus genome and further comprises a viral envelope comprising one or more mutated viral envelope glycoproteins that mediate fusion of the viral particle and the target host cell but that do not bind its cognate receptor expressed on the target host cell and one or more non-viral membrane-bound tropism polypeptides.

In particular embodiments, one or more mutated viral envelope glycoproteins comprise a vesiculovirus envelope glycoprotein or one or more morbillivirus envelope glycoproteins or henipavirus envelope glycoproteins. In particular embodiments, the mutated viral envelope glycoprotein comprises a heterologous signal peptide.

In particular embodiments, one or more mutated viral envelope glycoproteins comprise a vesiculovirus envelope glycoprotein or one or more morbillivirus envelope glycoproteins or henipavirus envelope glycoproteins.

Illustrative examples of vesiculoviruses suitable for use in particular embodiments from which G glycoproteins can be isolated from include, but are not limited to vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV, f.k.a. VSV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In particular embodiments, a vesiculovirus G envelope protein comprises one or more amino acid substitutions that that enable the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises an amino acid sequence set forth in any one of SEQ ID NOs: 15-322 disclosed in U.S. Patent Application No. 20200216502, each said sequence incorporated by reference herein in its entirety, or an amino acid sequence at least 95%, 96%, 97%, 98%, or 99% identical thereto that enables the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises amino acid substitution at positions 47 and/or 354 (or 358 in some strains, e.g., CJSV, VSNJV) with reference to the vesiculovirus G envelope protein lacking a signal peptide.

In particular embodiments, a vesiculovirus is vesicular stomatitis Indiana virus (VSIV). In particular embodiments, a mutated viral envelope glycoprotein is derived from a VSIV envelope glycoprotein (VSIV-G; e.g., SEQ ID NO: 1: KFTIVFPHNQKGNWKNVPSNYHYCPSSSDLNWHNDLIGTALQVKMPKSHKAIQA DGWMCHASKWVTTCDFRWYGPKYITHSIRSFTPSVEQCKESIEQTKQGTWLNPG FPPQSCGYATVTDAEAVIVQVTPHHVLVDEYTGEWVDSQFINGKCSNYICPTVHN STTWHSDYKVKGLCDSNLISMDITFFSEDGELSSLGKEGTGFRSNYFAYETGGKA CKMQYCKHWGVRLPSGVWFEMADKDLFAAARFPECPEGSSISAPSQTSVDVSLI QDVERILDYSLCQETWSKIRAGLPISPVDLSYLAPKNPGTGPAFTIINGTLKYFETR YIRVDIAAPILSRMVGMISGTTTERELWDDWAPYEDVEIGPNGVLRTSSGYKFPLY MIGHGMLDSDLHLSSKAQVFEHPHIQDAASQLPDDESLFFGDTGLSKNPIELVEG WFSSWKSSIASFFFIIGLIIGLFLVLRVGIHLCIKLKHTKKRQIYTDIEMNRLGK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a mutated viral envelope glycoprotein is derived from a VSIV-G polypeptide set forth in SEQ ID NO: 1 comprising L47I and/or H80Q amino acid substitutions, such substitutions being present in naturally occurring variants of VSIV.

In particular embodiments, a mutated VSIV-G envelope protein comprises one or more of: one or more amino acid substitutions at H8, N9, Q10, K47, K50, A51, S183, S179, N180, I182, M184, Y209, T214, I347, T350, T352, E353, and R354 (substitution with any amino acid; a conservation substitution; a disruptive substitution; substitution with D, E, A, G, F, or Q; or substitution with A, G, F, or Q); an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; or a deletion of residues 1-8. In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. Amino acid positions are with reference to a VSIV-G envelope protein that lacks a signal peptide, e.g., SEQ ID NO: 1. In particular embodiments, a mutated viral envelope glycoprotein is derived from a VSIV-G polypeptide set forth in SEQ ID NO: 1 comprising L47I and/or H80Q amino acid substitutions, such substitutions present in naturally occurring variants of VSIV.

In particular embodiments, a mutated VSIV-G polypeptide comprises one or more amino acid substitutions at K47, I182, and/or R354 (substitution with any amino acid; a conservation substitution; a disruptive substitution; substitution with D, E, A, G, F, or Q; or substitution with A, G, F, or Q). In particular embodiments, a mutated VSIV-G polypeptide comprises amino acid substitutions at K47, I182, or R354; K47 and I182; K47 and R354; I182 and R354; or at K47, I182, and R354 of SEQ ID NO: 1.

In particular embodiments, a mutated VSIV-G polypeptide comprises one or more of the following amino acid substitutions: K47A, K47Q, I182E, I182D, R354A, and/or R354Q. In particular embodiments, a mutated VSIV-G polypeptide comprises the following amino acid substitutions: K47A, K47Q, I182E, I182D, R354A, or R354Q; K47A and I1182E; K47A and I182D; K47Q and I182E; K47Q and I182D; I182E and R354A; I182E and R354Q; I182D and R354A; I182D and R354Q; K47A and R354A; K47A and R354Q; K47Q and R354A; K47Q and R354Q; K47A, I182E, and R354A; K47A, I182D, and R354A; K47Q, I182E, and R354A; K47Q, I182D, and R354A; K47A, I182E, and R354Q; K47A, I182D, and R354Q; K47Q, I182E, and R354Q; or K47Q, I182D, and R354Q of SEQ ID NO: 1.

In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. In particular embodiments, a VSIV-G envelope protein comprises one or more amino acid substitutions at K47 and R354 (with reference to a VSIV-G envelope protein that lacks a signal peptide, e.g., SEQ ID NO: 1). In particular embodiments, a VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In preferred embodiments, a VSIV-G envelope comprises the amino acid substitutions K47Q or K47A and R354A or R354Q. Amino acid positions are with reference to a VSIV-G envelope protein that lacks a signal peptide.

In particular embodiments, a VSIV-G envelope protein comprises an amino acid sequence set forth in SEQ ID NO: 2 (wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q) or an amino acid sequence at least 95%, 96%, 97%, 98%, or 99% identical thereto that mediates fusion of the viral particle and the target host cell but that does not bind its cognate receptor expressed on the target host cell, e.g., LDL-R.

TABLE 1

| SEQ ID | |
|---|---|
| NO | Amino Acid Sequence |
| 2 | KFTIVFPHNQKGNWKNVPSNYHYCPSSSDLNWHNDLIGTAX₁QVKMPX₂SHKAIQADGWMCHASKW |
| | VTTCDFRWYGPKYITX₃SIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSEDG |
| | ELSSLGKEGTGFRSNYFAYETGGKACKMQYCKHWGVRLPSGVWFEMADKDLFAAARFPECPEGSS |
| | ISAPSQTSVDVSLIQDVERILDYSLCQETWSKIRAGLPISPVDLSYLAPKNPGTGPAFTIINGTL |
| | KYFETRYIRVDIAAPILSRMVGMISGTTTEX₄ELWDDWAPYEDVEIGPNGVLRTSSGYKFPLYMI |
| | GHGMLDSDLHLSSKAQVFEHPHIQDAASQLPDDESLFFGDTGLSKNPIELVEGWFSSWKSSIASE |
| | FFIIGLIIGLFLVLRVGIHLCIKLKHTKKRQIYTDIEMNRLGK |

In particular embodiments, a mutated VSIV-G envelope protein comprises an amino acid sequence set forth in any one of SEQ ID NOs: 13-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61, 65, 69, and 73 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 2

| SEQ | |
|---|---|
| ID | |
| NO: | AMINO ACID SEQUENCE |
| 13 | KFTIVFPHNQKGNWKNVPSNYHYCPSSSDLNWHNDLIGTAIQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 14 | KFTIVFPHNQKGNWKNVPSNYHYCPSSSDLNWHNDLIGTAIQVKMPASHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 15 | KFTIVFPHNQKGNWKNVPSNYHYCPSSSDLNWHNDLIGTAIQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWESSWKSS |
| | IASFFFIIGLIIGLFLVLRVGIHLCIKLKHTKKRQIYTDIEMNRLGK |
| 16 | KFTIVFPHNQKGNWKNVPSNYHYCPSSSDLNWHNDLIGTAIQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 17 | KFTIVFPHNQKGNWKNVPSNYHYCPSSSDLNWHNDLIGTALQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 18 | KFTIVFPHNQKGNWKNVPSNYHYCPSSSDLNWHNDLIGTALQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 19 | KFTIVFPHNQKGNWKNVPSNYHYCPSSSDLNWHNDLIGTALQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 20 | KFTIVFPHNQKGNWKNVPSNYHYCPSSSDLNWHNDLIGTALQVKMPASHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 21 | KFTIVFPHNQKGNWKNVPSNYHYCPSSSDLNWHNDLIGTAIQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 22 | KFTIVFPHNQKGNWKNVPSNYHYCPSSSDLNWHNDLIGTAIQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 23 | KFTIVFPHNQKGNWKNVPSNYHYCPSSSDLNWHNDLIGTAIQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 24 | KFTIVFPHNQKGNWKNVPSNYHYCPSSSDLNWHNDLIGTAIQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 25 | KFTIVFPHNQKGNWKNVPSNYHYCPSSSDLNWHNDLIGTALQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 26 | KFTIVFPHNQKGNWKNVPSNYHYCPSSSDLNWHNDLIGTALQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 27 | KFTIVFPHNQKGNWKNVPSNYHYCPSSSDLNWHNDLIGTALQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 28 | KFTIVFPHNQKGNWKNVPSNYHYCPSSSDLNWHNDLIGTALQVKMPASHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 29 | KFTIVFPHNQKGNWKNVPSNYHYCPSSSDLNWHNDLIGTAIQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 30 | KFTIVFPHNQKGNWKNVPSNYHYCPSSSDLNWHNDLIGTAIQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 31 | KFTIVFPHNQKGNWKNVPSNYHYCPSSSDLNWHNDLIGTAIQVKMPGSHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 32 | KFTIVFPHNQKGNWKNVPSNYHYCPSSSDLNWHNDLIGTAIQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 33 | KFTIVFPHNQKGNWKNVPSNYHYCPSSSDLNWHNDLIGTALQVKMPGSHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 34 | KFTIVFPHNQKGNWKNVPSNYHYCPSSSDLNWHNDLIGTALQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWESSWKSS |
| | IASFFFIIGLIIGLFLVLRVGIHLCIKLKHTKKRQIYTDIEMNRLGK |
| 35 | KFTIVFPHNQKGNWKNVPSNYHYCPSSSDLNWHNDLIGTALQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 36 | KFTIVFPHNQKGNWKNVPSNYHYCPSSSDLNWHNDLIGTALQVKMPGSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 37 | KFTIVFPHNQKGNWKNVPSNYHYCPSSSDLNWHNDLIGTAIQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 38 | KFTIVFPHNQKGNWKNVPSNYHYCPSSSDLNWHNDLIGTAIQVKMPGSHKAIQADGWMCHASKW |
| | VTTCDFRWYGPKYITHSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 39 | KFTIVFPHNQKGNWKNVPSNYHYCPSSSDLNWHNDLIGTAIQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 40 | KFTIVFPHNQKGNWKNVPSNYHYCPSSSDLNWHNDLIGTAIQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 41 | KFTIVFPHNQKGNWKNVPSNYHYCPSSSDLNWHNDLIGTALQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 42 | KFTIVFPHNQKGNWKNVPSNYHYCPSSSDLNWHNDLIGTALQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 43 | KFTIVFPHNQKGNWKNVPSNYHYCPSSSDLNWHNDLIGTALQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKEPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 44 | KFTIVFPHNQKGNWKNVPSNYHYCPSSSDLNWHNDLIGTAIQVKMPGSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 45 | KFTIVFPHNQKGNWKNVPSNYHYCPSSSDLNWHNDLIGTAIQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 46 | KFTIVFPHNQKGNWKNVPSNYHYCPSSSDLNWHNDLIGTAIQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 47 | KFTIVFPHNQKGNWKNVPSNYHYCPSSSDLNWHNDLIGTAIQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 48 | KFTIVFPHNQKGNWKNVPSNYHYCPSSSDLNWHNDLIGTAIQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKEPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 49 | KFTIVFPHNQKGNWKNVPSNYHYCPSSSDLNWHNDLIGTALQVKMPFSHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 50 | KFTIVFPHNQKGNWKNVPSNYHYCPSSSDLNWHNDLIGTALQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 51 | KFTIVFPHNQKGNWKNVPSNYHYCPSSSDLNWHNDLIGTALQVKMPFSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 52 | KFTIVFPHNQKGNWKNVPSNYHYCPSSSDLNWHNDLIGTALQVKMPFSHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 53 | KFTIVFPHNQKGNWKNVPSNYHYCPSSSDLNWHNDLIGTAIQVKMPFSHKAIQADGWMCHASKW |
| | VTTCDFRWYGPKYITHSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 54 | KFTIVFPHNQKGNWKNVPSNYHYCPSSSDLNWHNDLIGTAIQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 55 | KFTIVFPHNQKGNWKNVPSNYHYCPSSSDLNWHNDLIGTAIQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKEPL |
| | YMIGHGMLDSDLHLSSKAQVFEHPHIQDAASQLPDDESLFFGDTGLSKNPIELVEGWESSWKSS |
| | IASFFFIIGLIIGLFLVLRVGIHLCIKLKHTKKRQIYTDIEMNRLGK |
| 56 | KFTIVFPHNQKGNWKNVPSNYHYCPSSSDLNWHNDLIGTAIQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 57 | KFTIVFPHNQKGNWKNVPSNYHYCPSSSDLNWHNDLIGTALQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 58 | KFTIVFPHNQKGNWKNVPSNYHYCPSSSDLNWHNDLIGTALQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 59 | KFTIVFPHNQKGNWKNVPSNYHYCPSSSDLNWHNDLIGTALQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKEPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 60 | KFTIVFPHNQKGNWKNVPSNYHYCPSSSDLNWHNDLIGTALQVKMPFSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 61 | KFTIVFPHNQKGNWKNVPSNYHYCPSSSDLNWHNDLIGTAIQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 62 | KFTIVFPHNQKGNWKNVPSNYHYCPSSSDLNWHNDLIGTAIQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 63 | KFTIVFPHNQKGNWKNVPSNYHYCPSSSDLNWHNDLIGTAIQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 64 | KFTIVFPHNQKGNWKNVPSNYHYCPSSSDLNWHNDLIGTAIQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 65 | KFTIVFPHNQKGNWKNVPSNYHYCPSSSDLNWHNDLIGTALQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWESSWKSS |
| | IASFFFIIGLIIGLFLVLRVGIHLCIKLKHTKKRQIYTDIEMNRLGK |
| 66 | KFTIVFPHNQKGNWKNVPSNYHYCPSSSDLNWHNDLIGTALQVKMPQSHKAIQADGWMCHASKW |
| | VTTCDFRWYGPKYITQSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 67 | KFTIVFPHNQKGNWKNVPSNYHYCPSSSDLNWHNDLIGTALQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 68 | KFTIVFPHNQKGNWKNVPSNYHYCPSSSDLNWHNDLIGTALQVKMPQSHKAIQADGWMCHASKW |
| | VTTCDFRWYGPKYITQSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 69 | KFTIVFPHNQKGNWKNVPSNYHYCPSSSDLNWHNDLIGTAIQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 70 | KFTIVFPHNQKGNWKNVPSNYHYCPSSSDLNWHNDLIGTAIQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 71 | KFTIVFPHNQKGNWKNVPSNYHYCPSSSDLNWHNDLIGTAIQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 72 | KFTIVFPHNQKGNWKNVPSNYHYCPSSSDLNWHNDLIGTAIQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 73 | KFTIVFPHNQKGNWKNVPSNYHYCPSSSDLNWHNDLIGTALQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEAELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 74 | KFTIVFPHNQKGNWKNVPSNYHYCPSSSDLNWHNDLIGTALQVKMPQSHKAIQADGWMCHASKW |
| | VTTCDFRWYGPKYITHSIRSFTPSVEQCKESIEQTKQGTWLNPGFPPQSCGYATVIDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEGELWDDWAPYEDVEIGPNGVLRTSSGYKFPL |
| | YMIGHGMLDSDLHLSSKAQVFEHPHIQDAASQLPDDESLFFGDTGLSKNPIELVEGWESSWKSS |
| | IASFFFIIGLIIGLFLVLRVGIHLCIKLKHTKKRQIYTDIEMNRLGK |
| 75 | KFTIVFPHNQKGNWKNVPSNYHYCPSSSDLNWHNDLIGTALQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEFELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |
| 76 | KFTIVFPHNQKGNWKNVPSNYHYCPSSSDLNWHNDLIGTALQVKMPQSHKAIQADGWMCHASKW |
| | VTTCDFRWYGPKYITHSIRSFTPSVEQCKESIEQTKQGTWLNPGFPPQSCGYATVTDAEAVIVQ |
| | VTPHHVLVDEYTGEWVDSQFINGKCSNYICPTVHNSTTWHSDYKVKGLCDSNLISMDITFFSED |
| | GELSSLGKEGTGFRSNYFAYETGGKACKMQYCKHWGVRLPSGVWFEMADKDLFAAARFPECPEG |
| | SSISAPSQTSVDVSLIQDVERILDYSLCQETWSKIRAGLPISPVDLSYLAPKNPGTGPAFTIIN |
| | GTLKYFETRYIRVDIAAPILSRMVGMISGTTTEQELWDDWAPYEDVEIGPNGVLRTSSGYKFPL |
| | YMIGHGMLDSDLHLSSKAQVFEHPHIQDAASQLPDDESLFFGDTGLSKNPIELVEGWFSSWKSS |
| | IASFFFIIGLIIGLFLVLRVGIHLCIKLKHTKKRQIYTDIEMNRLGK |

In particular embodiments, a vesiculovirus is cocal virus (COCV). In particular embodiments, a mutated viral envelope glycoprotein is derived from a COCV envelope glycoprotein (COCV-G; e.g., SEQ ID NO: 3: KFSIVFPQSQKGNWKNVPSSYHYCP SSSDQNWHNDLLGITMKVKMPKTHKAIQADGWMCHAAKWITTCDFRWYGPKY ITHSIHSIQPTSEQCKESIKQTKQGTWMSPGFPPQNCGYATVTDSVAVVVQATPHH VLVDEYTGEWIDSQFPNGKCETEECETVHNSTVWYSDYKVTGLCDATLVDTEITF FSEDGKKESIGKPNTGYRSNYFAYEKGDKVCKMNYCKHAGVRLPSGVWFEFVD QDVYAAAKLPECPVGATISAPTQTSVDVSLILDVERILDYSLCQETWSKIRSKQPV SPVDLSYLAPKNPGTGPAFTIINGTLKYFETRYIRIDIDNPIISKMVGKISGSQTEREL WTEWFPYEGVEIGPNGILKTPTGYKFPLFMIGHGMLDSDLHKTSQAEVFEHPHLA EAPKQLPEEETLFFGDTGISKNPVELIEGWFSSWKSTVVTFFFAIGVFILLYVVARI VIAVRYRYQGSNNKRIYNDIEMSRFRK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a COCV-G envelope protein comprises one or more amino acid substitutions at K47 and/or R354. In particular embodiments, a COCV-G envelope protein comprises one or more amino acid substitutions at K47 and/or R354, wherein each amino acid can be substituted with A, G, F, or Q. In particular embodiments, a COCV-G envelope protein comprises one or more amino acid substitutions at K47 and/or R354 (with reference to a COCV-G envelope protein that lacks a signal peptide, e.g., SEQ ID NO: 3). In particular embodiments, a COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In particular embodiments, a COCV-G envelope protein comprises an amino acid sequence set forth in SEQ ID NO: 4 (wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=A and X₂=Q) or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 3

| SEQ ID | |
|---|---|
| NO | Amino Acid Sequence |
| 4 | KFSIVFPQSQKGNWKNVPSSYHYCPSSSDQ |
| | NWHNDLLGITMKVKMPX₁THKAIQADGWMC |
| | HAAKWITTCDFRWYGPKYITHSIHSIQPTS |
| | EQCKESIKQTKQGTWMSPGFPPQNCGYATV |
| | TDSVAVVVQATPHHVLVDEYTGEWIDSQFP |
| | NGKCETEECETVHNSTVWYSDYKVTGLCDA |
| | TLVDTEITFFSEDGKKESIGKPNTGYRSNY |
| | FAYEKGDKVCKMNYCKHAGVRLPSGVWFEF |
| | VDQDVYAAAKLPECPVGATISAPTQTSVDV |
| | SLILDVERILDYSLCQETWSKIRSKQPVSP |
| | VDLSYLAPKNPGTGPAFTIINGTLKYFETR |
| | YIRIDIDNPIISKMVGKISGSQTEX₁ELWT |
| | EWFPYEGVEIGPNGILKTPTGYKFPLFMIG |
| | HGMLDSDLHKTSQAEVFEHPHLAEAPKQLP |
| | EEETLFFGDTGISKNPVELIEGWFSSWKST |
| | VVTFFFAIGVFILLYVVARIVIAVRYRYQG |
| | SNNKRIYNDIEMSRERK |

In particular embodiments, a mutated COCV-G envelope protein comprises an amino acid sequence set forth in any one of SEQ ID NOs: 77-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated COCV-G envelope protein comprises an amino acid sequence set forth in any one of SEQ ID NOs: 89-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in SEQ ID NOs: 89 or 92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 4

| SEQ | |
|---|---|
| ID | |
| NO: | AMINO ACID SEQUENCE |
| 77 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 78 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEGELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 79 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEFELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 80 | KFSIVFPQSQKGNWKNVPSSYHYCPSSSDQNWHNDLLGITMKVKMPATHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 81 | KFSIVFPQSQKGNWKNVPSSYHYCPSSSDQNWHNDLLGITMKVKMPGTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 82 | KFSIVFPQSQKGNWKNVPSSYHYCPSSSDQNWHNDLLGITMKVKMPGTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEGELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 83 | KFSIVFPQSQKGNWKNVPSSYHYCPSSSDQNWHNDLLGITMKVKMPGTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEFELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 84 | KFSIVFPQSQKGNWKNVPSSYHYCPSSSDQNWHNDLLGITMKVKMPGTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 85 | KFSIVFPQSQKGNWKNVPSSYHYCPSSSDQNWHNDLLGITMKVKMPFTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 86 | KFSIVFPQSQKGNWKNVPSSYHYCPSSSDQNWHNDLLGITMKVKMPFTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEGELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 87 | KFSIVFPQSQKGNWKNVPSSYHYCPSSSDQNWHNDLLGITMKVKMPFTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEFELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 88 | KFSIVFPQSQKGNWKNVPSSYHYCPSSSDQNWHNDLLGITMKVKMPFTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 89 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEAELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 90 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEGELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 91 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEFELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |
| 92 | KFSIVFPQSQKGNWKNVPSSYHYCPSSSDQNWHNDLLGITMKVKMPQTHKAIQADGWMCHAAKW |
| | ITTCDFRWYGPKYITHSIHSIQPTSEQCKESIKQTKQGTWMSPGFPPQNCGYATVTDSVAVVVQ |
| | ATPHHVLVDEYTGEWIDSQFPNGKCETEECETVHNSTVWYSDYKVTGLCDATLVDTEITFFSED |
| | GKKESIGKPNTGYRSNYFAYEKGDKVCKMNYCKHAGVRLPSGVWFEFVDQDVYAAAKLPECPVG |
| | ATISAPTQTSVDVSLILDVERILDYSLCQETWSKIRSKQPVSPVDLSYLAPKNPGTGPAFTIIN |
| | GTLKYFETRYIRIDIDNPIISKMVGKISGSQTEQELWTEWFPYEGVEIGPNGILKTPTGYKFPL |
| | FMIGHGMLDSDLHKTSQAEVFEHPHLAEAPKQLPEEETLFFGDTGISKNPVELIEGWFSSWKST |
| | VVTFFFAIGVFILLYVVARIVIAVRYRYQGSNNKRIYNDIEMSRFRK |

In particular embodiments, one or more mutated morbillivirus envelope glycoproteins are derived from measles virus F (MV-F) polypeptide (e.g., SEQ ID NO: 5: QIHWGNLSKIGVVGIGSASYKVMTRSSHQSLVIKLMPNITLLNNCTRVEIAEYRRL LRTVLEPIRDALNAMTQNIRPVQSVASSRRHKRFAGVVLAGAALGVATAAQITA GIALHQSMLNSQAIDNLRASLETTNQAIEAIRQAGQEMILAVQGVQDYINNELIPS MNQLSCDLIGQKLGLKLLRYYTEILSLFGPSLRDPISAEISIQALSYALGGDINKVLE KLGYSGGDLLGILESRGIKARITHVDTESYFIVLSIAYPTLSEIKGVIVHRLEGVSYN IGSQEWYTTVPKYVATQGYLISNFDESSCTFMPEGTVCSQNALYPMSPLLQECLR GSTKSCARTLVSGSFGNRFILSQGNLIANCASILCKCYTTGTIINQDPDKILTYIAAD HCPVVEVNGVTIQVGSRRYPDAVYLHRIDLGPPISLERLDVGTNLGNAIAKLEDA KELLESSDQILRSMKGLSSTSIVYILIAVCLGGLIGIPALICCCRGR) or an amino acid sequence at least 95%, 96%, 97%, 98%, or 99% identical thereto and a measles virus H (MV-H) polypeptide (e.g., SEQ ID NO: 6: MGSRIVINREHLMIDRPYVLLAVLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETCFQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPYLFTVPIKEAGGDCHAPTYLPAEVDGDVKLSSNLVI LPGQDLQYVLATYDTSRVEHAVVYYVYSPSRSFSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto, wherein the MV-H protein lacks cell binding activity and retains fusogenic activity. In particular embodiments, the MV-H polypeptide comprises one or more amino acid substitutions at positions Y463, R515, S530, and F531 of a MV-H polypeptide (e.g., SEQ ID NO 6). In particular embodiments, the MV-H polypeptide comprises one or more of the amino acid substitutions Y463A, R515A, S530L, and F531S in an MV-H polypeptide (e.g., SEQ ID NO: 7: MGSRIVINREHLMIDRPYVLLAVLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETCFQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPALFNVPIKEAGGDCHAPTYLPAEVDGDVKLSSNLVI LPGQDLQYVLATYDTSAVEHAVVYYVYSPSRLSSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto.

In particular embodiments, one or more mutated henipavirus envelope glycoproteins are derived from a nipah virus F (NiV-F) polypeptide (e.g., SEQ ID NO: 8: LHYEKLSKIGLVKGVTRKYKIKSNPLTKDIVIKMIPNVSNMSQCTGSVMENYKTR LNGILTPIKGALEIYKNNTHDLVGDVRLAGVIMAGVAIGIATAAQITAGVALYEA MKNADNINKLKSSIESTNEAVVKLQETAEKTVYVLTALQDYINTNLVPTIDKISCK QTELSLDLALSKYLSDLLFVFGPNLQDPVSNSMTIQAISQAFGGNYETLLRTLGYA TEDFDDLLESDSITGQIIYVDLSSYYIIVRVYFPILTEIQQAYIQELLPVSFNNDNSEW ISIVPNFILVRNTLISNIEIGFCLITKRSVICNQDYATPMTNNMRECLTGSTEKCPREL VVSSHVPRFALSNGVLFANCISVTCQCQTTGRAISQSGEQTLLMIDNTTCPTAVLG NVIISLGKYLGSVNYNSEGIAIGPPVFTDKVDISSQISSMNQSLQQSKDYIKEAQRL LDTVNPSLISMLSMIILYVLSIASLCIGLITFISFIIVEKKRNT) or an amino acid sequence at least 95%, 96%, 97%, 98%, or 99% identical thereto and a nipah virus G (NiV-G) polypeptide (e.g., SEQ ID NO: 9: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPEICWEGVYNDAFLIDRINWISAGVFLDSNQTAENPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity. In particular embodiments, the NiV-G polypeptide comprises one or more amino acid substitutions at positions E468, W471, Q497, and E500 of an NiV-G polypeptide (e.g., SEQ ID NO: 9). In particular embodiments, the NiV-G polypeptide comprises one or more of the amino acid substitutions E468A, W471A, Q497A, and E500A in an NiV-G polypeptide (e.g., SEQ ID NO: 10: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPAICAEGVYNDAFLIDRINWISAGVFLDSNATAANPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity.

In particular embodiments, a recombinant retrovirus contemplated herein comprises an outer surface, a lipid bilayer, cell membrane, or viral envelope comprising or expressing one or more non-viral membrane bound tropism polypeptides. A “tropism polypeptide” is a polypeptide that binds one or more antigens on a target host cell. A “non-viral membrane bound tropism polypeptide” is a polypeptide that binds one or more antigens on a target host cell; that is not native to, or derived from, either in whole or in part, a virus; and that is attached to a lipid bilayer, cell membrane, or viral envelope. In particular embodiments, a recombinant retrovirus contemplated herein comprises a viral envelope comprising or expressing a primary non-viral membrane bound tropism polypeptide and a secondary non-viral membrane bound tropism polypeptide.

In particular embodiments, a non-viral membrane bound tropism polypeptide comprises an extracellular antigen targeting domain, a spacer domain, and a transmembrane domain. In particular embodiments, a non-viral membrane bound tropism polypeptide comprises from N-terminus to C-terminus, an extracellular antigen targeting domain, a spacer domain, and a transmembrane domain. A “spacer domain” or “spacer polypeptide” refers to a polypeptide disposed between an extracellular antigen targeting domain and a transmembrane domain. A spacer domain positions the extracellular antigen targeting domain away from the viral envelope surface to enable proper virus/target cell contact and binding. A spacer domain may be derived either from a natural, synthetic, semi-synthetic, or recombinant source. Illustrative examples of spacer domains include but are not limited to hinge or stalk domains from IgG1, IgG2, IgG4, CD2, CD3, CD4, CD8α, CD8β, and CD28 and polypeptide linkers of similar amino acid composition and lengths.

In particular embodiments, an extracellular antigen targeting domain binds an antigen expressed on an immune effector cell. In particular embodiments, an extracellular antigen targeting domain comprises an antibody or antigen binding fragment thereof that binds an antigen expressed on the surface of an immune effector cell. In particular embodiments, an extracellular antigen targeting domain binds an antigen expressed on an immune effector cell.

In particular embodiments, the extracellular antigen targeting domain binds CD3δ, CD3ε, CD3γ, CD4, CD5, CD7, CD8α, or CD8β. In particular embodiments, the extracellular antigen targeting domain comprises an scFv, a murine scFv, a humanized scFv, or a human scFv that binds CD3δ, CD3ε, CD3γ, CD4, CD5, CD7, CD8α, or CD8β.

In particular embodiments, a tropism polypeptide comprises an anti-CD3p antibody or antigen binding fragment thereof. Illustrative examples of anti-CD3p antibodies or antigen binding fragments thereof suitable for using in particular embodiments include scFvs or other antigen binding fragments isolated from OKT3, UCHT1, YTH12.5, and TR66, and variants thereof, e.g., teplizumab and variants having at least 75%, at least 80%, at least 85%, at least 90%, at least 95%, or at least 99% identity thereto.

Illustrative anti-CD3 scFvs include the following amino acid sequences.

TABLE 5

| SEQ ID | |
|---|---|
| NO: | Amino Acid Sequence |
| 11 | DIQMTQSPSSLSASVGDRVTITCSASSSVS |
| | YMNWYQQTPGKAPKRWIYDTSKLASGVPSR |
| | FSGSGSGTDYTFTISSLQPEDIATYYCQQW |
| | SSNPFTFGQGTKLQITRTSGGGGSGGGGSG |
| | GGGSQVQLVQSGGGVVQPGRSLRLSCKASG |
| | YTFTRYTMHWVRQAPGKGLEWIGYINPSRG |
| | YTNYNQKVKDRFTISRDNSKNTAFLQMDSL |
| | RPEDTGVYFCARYYDDHYCLDYWGQGTPVT |
| | VSS |
| 12 | DIQMTQSPSSLSASVGDRVTITCSASSSVS |
| | YMNWYQQTPGKAPKRWIYDTSKLASGVPSR |
| | FSGSGSGTDYTFTISSLQPEDIATYYCQQW |
| | SSNPFTFGQGTKLQITRTSGGGGSGGGGSG |
| | GGGSQVQLVQSGGGVVQPGRSLRLSCKASG |
| | YTFTRYTMHWVRQAPGKGLEWIGYINPSRG |
| | YTNYNQKVKDRFTISRDNSKNTAFLQMDSL |
| | RPEDTGVYFCARYYDDHYCLDYWGQGTPVT |
| | VSS |

In particular embodiments, a tropism polypeptide comprises an anti-CD3 scFv, a spacer domain and a transmembrane domain (e.g., isolated from CD3, CD4, CD8α, CD28, or Glycophorin A), and a cytoplasmic tail (e.g., 1, 2, 3, 4, 5 or more amino acids).

In particular embodiments, a recombinant retrovirus contemplated herein comprises a viral envelope comprising or expressing a primary non-viral membrane bound tropism polypeptide comprising an extracellular antigen targeting domain binds CD3δ, CD3ε, CD3γ, CD4, CD5, CD7, CD8α, or CD8β, a spacer domain, and a transmembrane domain; and further comprises a secondary non-viral membrane bound tropism polypeptide.

In particular embodiments, a secondary non-viral membrane bound tropism polypeptide comprises an extracellular antigen targeting domain that binds a T cell surface antigen including but not limited to the alpha or beta chains of a TCR, CD28, CD134 (OX40), CD137 (4-1BB), and CD278 (ICOS).

In particular embodiments, a secondary non-viral membrane bound tropism polypeptide comprises an extracellular antigen targeting domain comprising an antibody or antigen binding fragment thereof that specifically binds a T cell surface antigen including but not limited to the alpha or beta chains of a TCR, CD28, CD134 (OX40), CD137 (4-1BB), and CD278 (ICOS).

In particular embodiments, a secondary non-viral membrane bound tropism polypeptide comprises all or part of a co-stimulatory molecule including but not limited to CD80, CD86, OX40L, 4-1BBL, and ICOSL. In particular embodiments, the functional fragment is a CD80, CD86, OX40L, 4-1BBL, and ICOSL comprising a C-terminal truncation that results in a cytoplasmic domain of about 1, 2, 3, 4, 5, 6, 7, 8, 9, or 10 amino acids or more).

In particular embodiments, a recombinant retrovirus contemplated herein comprises a viral envelope comprising or expressing a primary non-viral membrane bound tropism polypeptide comprising an extracellular antigen targeting domain binds CD3δ, CD3ε CD3γ, CD4, CD8α, or CD8β, a spacer domain, and a transmembrane domain; and further comprises a secondary non-viral membrane bound tropism polypeptide comprising all or part of a co-stimulatory molecule including but not limited to CD80, CD86, OX40L, 4-1BBL, and ICOSL or a functional fragment thereof, e.g., a CD80, CD86, OX40L, 4-1BBL, and ICOSL, comprising a C-terminal truncation that results in a cytoplasmic domain of about 1, 2, 3, 4, 5, 6, 7, 8, 9, or 10 amino acids or more).

### E. Engineered Antigen Receptors

In particular embodiments, a recombinant retrovirus contemplated herein comprises a retroviral vector comprising a polynucleotide encoding a promoter operably linked to a polynucleotide encoding one or more engineered antigen receptors. Engineered antigen receptors include but are not limited to T cell receptors (TCRs), chimeric antigen receptors (CARs) and dimerization activated receptor initiation complexes (DARICs).

In particular embodiments, the engineered antigen receptor is designed to redirect an immune effector cell to bind or target an antigen on a target cell, e.g., a cancer cell. In particular embodiments, the engineered antigen receptor comprises an extracellular antigen binding domain that binds to alpha folate receptor (FRα), αvβ6 integrin, B cell maturation antigen (BCMA), B7-H3 (CD276), B7-H6, carbonic anhydrase IX (CAIX), CD16, CD 19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant IP (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), G Protein-Coupled Receptor Class C Group 5 Member D (GPCR5D), EGFR family including ErbB2 (HER2), IL-10Rα, IL-13Rα2, Kappa, cancer/testis antigen 2 (LAGE-1A), Lambda, Lewis-Y (LeY), LI cell adhesion molecule (L1-CAM), melanoma antigen gene (MAGE)-A1, MAGE-A3, MAGE-A4, MAGE-A6, MAGE A10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL 16-binding protein (ULBP) 1, ULBP2, ULBP3, ULBP4, ULBP5, ULBP6, vascular endothelial growth factor receptor 2 (VEGFR2), or Wilms tumor 1 (WT-1).

### 1. T Cell Receptors

In particular embodiments, a retroviral vector comprises a polynucleotide encoding a promoter operably linked to a polynucleotide encoding a TCR. Naturally occurring TCRs comprise two subunits, an alpha chain and a beta chain subunit (αβTCR), or a gamma chain and a delta chain subunit (γδTCR), each of which is a unique protein produced by a recombination event in each T cell's genome. In one embodiment, the TCR is an αβTCR. In one embodiment, the TCR is a γδTCR.

In particular embodiments, polynucleotides encoding TCRs are preferably isolated from their natural context in a (naturally-occurring) chromosome of a T cell and incorporated into a retroviral vector contemplated herein. In particular embodiments, the TCR is an exogenous TCR because it is introduced into immune effector cells that do not normally express the particular TCR. The essential aspect of the TCRs is that it has high avidity for a tumor antigen presented by a major histocompatibility complex (MHC) or similar immunological component. In contrast to TCRs, CARs are engineered to bind target antigens in an MHC independent manner.

A TCR can be expressed with additional polypeptides attached to the amino-terminal or carboxyl-terminal portion of the alpha chain or beta chain of a TCR, or of the gamma chain or delta chain of a TCR so long as the attached additional polypeptide does not interfere with the ability of the chains to form a functional T cell receptor and recognize antigen.

Antigens that are recognized by the engineered TCRs contemplated in particular embodiments include, but are not limited to cancer antigens, including antigens on both hematological cancers and solid tumors. Illustrative antigens include, but are not limited to FRα, αvβ6 integrin, BCMA, CD276, B7-H6, CAIX, CD16, CD 19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvIII, EGP2, EGP40, EPCAM, EPHA2, FAP, FCRL5, AchR, GD2, GD3, GPC3, GPCR5D, HER2, IL-10Rα, IL-13Rα2, LAGE-1A, Lambda, LeY, L1-CAM, MAGE-A1, MAGE-A3, MAGE-A4, MAGE-A6, MAGE A10, MelanA or MARTI, MSLN, MUC1, MUC16, MICA, MICB, NCAM, NY-ESO-1, PLAC1, PRAME, PSCA, PSMA, ROR1, SSX2, Survivin, TAG72, TEM1, TEM7R, TPBG, ULBP1, ULBP2, ULBP3, ULBP4, ULBP5, ULBP6, VEGFR2, or WT-1.

### 2. Chimeric Antigen Receptors

In particular embodiments, a retroviral vector comprises a polynucleotide encoding a promoter operably linked to a polynucleotide encoding a CAR. A CAR is a molecule that combines antibody-based specificity for a target antigen with a T cell receptor-activating intracellular domain to generate a chimeric protein that exhibits a specific anti-tumor cellular immune activity.

In various embodiments, a CAR comprises an extracellular antigen binding domain that binds to a specific target antigen, a transmembrane domain and one or more intracellular signaling domains.

An antigen binding domain includes any naturally occurring, synthetic, semi-synthetic, or recombinantly produced binding partner for a biological molecule of interest. In particular embodiments, the extracellular binding domain comprises an antibody or antigen binding fragment thereof. In particular embodiments, the extracellular binding domain comprises an antibody or antigen binding fragment thereof is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), a single-domain antibody (sdAb, a camelid VHH, Nanobody) and a centyrin. In one embodiment, the binding domain comprises an scFv. In one embodiment, the binding domain comprises one or more VHH antibodies.

In particular embodiments, the CAR comprises an extracellular domain that binds an antigen selected from the group consisting of: FRα, αvβ6 integrin, BCMA, CD276, B7-H6, CAIX, CD16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvIII, EGP2, EGP40, EPCAM, EPHA2, FAP, FCRL5, AchR, GD2, GD3, GPC3, GPCR5D, HER2, IL-11Rα, IL-13Rα2, LAGE-1A, Lambda, LeY, L1-CAM, MAGE-A1, MAGE-A3, MAGE-A4, MAGE-A6, MAGEA10, MelanA or MART1, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

In particular embodiments, a CAR comprises a hinge domain. In particular embodiments, the hinge domain comprises the CH2 and CH3 domains of IgG1, IgG4, or IgD. Illustrative hinge domains suitable for use in the CARs described herein include the hinge region derived from the extracellular regions of type 1 membrane proteins such as CD28, CD8α, and CD4, which may be wild-type hinge regions from these molecules or may be altered.

The transmembrane (TM) domain of the CAR fuses the extracellular binding portion and intracellular signaling domain and anchors the CAR to the plasma membrane of the immune effector cell. The TM domain may be derived either from a natural, synthetic, semi-synthetic, or recombinant source. Illustrative TM domains may be derived from (i.e., comprise) at least the transmembrane region(s) of the alpha, beta, gamma, or delta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN, and PD-1. In particular embodiments, a CAR contemplated herein comprises a TM domain and a short oligo- or polypeptide linker, preferably between 1, 2, 3, 4, 5, 6, 7, 8, 9, or 10 amino acids in length that links the TM domain and the adjacent intracellular signaling domain of the CAR. A glycine-serine linker provides a particularly suitable linker.

In particular embodiments, a CAR comprises an intracellular signaling domain that comprises one or more “co-stimulatory signaling domains” and a “primary signaling domain.”

Primary signaling domains that act in a stimulatory manner may contain signaling motifs which are known as immunoreceptor tyrosine-based activation motifs or ITAMs.

Illustrative examples of ITAM containing primary signaling domains suitable for use in CARs contemplated in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more co-stimulatory signaling domains.

Illustrative examples of co-stimulatory domains suitable for use in CARs contemplated in particular embodiments include those isolated from TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In particular embodiments, a CAR comprises one or more co-stimulatory signaling domains selected from the group consisting of CD28, CD137, and CD134, and a CD3ζ primary signaling domain.

In various embodiments, a CAR comprises: an extracellular domain that binds an antigen selected from the group consisting of: BCMA, CD19, CD20, CD22, CD33, CD38, CD79a, CD79b, CD123, GPCR5D, MUC16 or mesothelin; a hinge domain; a transmembrane domain isolated from a polypeptide selected from the group consisting of: CD28, CD4, CD8α, CD154, and PD-1; one or more intracellular co-stimulatory signaling domains isolated from a polypeptide selected from the group consisting of: CD28, CD134, and CD137; and a signaling domain isolated from a polypeptide selected from the group consisting of: FcRγ, FcRβ, CD3γ, CD3δ, CD3ε, CD3ζ, CD22, CD79a, CD79b, and CD66d.

In particular embodiments, a CAR comprises an amino acid sequence set forth in any one of SEQ ID NOs: 93-97.

TABLE 6

| SEQ ID | |
|---|---|
| NO: | Amino Acid Sequence |
| 93 | DIVLTQSPPSLAMSLGKRATISCRASESVTILGSHLIHWYQQKPGQPPTLLIQLASNVQTGVPARFS |
| | GSGSRTDFTLTIDPVEEDDVAVYYCLQSRTIPRTFGGGTKLEIKGSTSGSGKPGSGEGSTKGQIQLV |
| | QSGPELKKPGETVKISCKASGYTFTDYSINWVKRAPGKGLKWMGWINTETREPAYAYDERGRFAFSL |
| | ETSASTAYLQINNLKYEDTATYFCALDYSYAMDYWGQGTSVTVSSAAATTTPAPRPPTPAPTIASQP |
| | LSLRPEACRPAAGGAVHTRGLDFACDIYIWAPLAGTCGVLLLSLVITLYCKRGRKKLLYIFKQPFMR |
| | PVQTTQEEDGCSCRFPEEEEGGCELRVKFSRSADAPAYQQGQNQLYNELNLGRREEYDVLDKRRGRD |
| | PEMGGKPRRKNPQEGLYNELQKDKMAEAYSEIGMKGERRRGKGHDGLYQGLSTATKDTYDALHMQAL |
| | PPR |
| 94 | DIQMTQTTSSLSASLGDRVTISCRASQDISKYLNWYQQKPDGTVKLLIYHTSRLHSGVPSRFSGSGS |
| | GTDYSLTISNLEQEDIATYFCQQGNTLPYTFGGGTKLEITGSTSGSGKPGSGEGSTKGEVKLQESGP |
| | GLVAPSQSLSVTCTVSGVSLPDYGVSWIRQPPRKGLEWLGVIWGSETTYYNSALKSRLTIIKDNSKS |
| | QVFLKMNSLQTDDTAIYYCAKHYYYGGSYAMDYWGQGTSVTVSSESKYGPPCPPCPMFWVLVVVGGV |
| | LACYSLLVTVAFIIFWVKRGRKKLLYIFKQPFMRPVQTTQEEDGCSCRFEEEEGGCELRVKFSRSAD |
| | APAYQQGQNQLYNELNLGRREEYDVLDKRRGRDPEMGGKPRRKNPQEGLYNELQKDKMAEAYSEIGM |
| | KGERRRGKGHDGLYQGLSTATKDTYDALHMQALPPR |
| 95 | QVKLEESGGGLVQAGRSLRLSCAASEHTFSSHVMGWFRQAPGKERESVAVIGWRDISTSYADSVKGR |
| | FTISRDNAKKTLYLQMNSLKPEDTAVYYCAARRIDAADFDSWGQGTQVTVSSGGGGSEVQLVESGGG |
| | LVQAGGSLRLSCAASGRTFTMGWFRQAPGKEREFVAAISLSPTLAYYAESVKGRFTISRDNAKNTVV |
| | LQMNSLKPEDTALYYCAADRKSVMSIRPDYWGQGTQVTVSSTSTTTPAPRPPTPAPTIASQPLSLRP |
| | EACRPAAGGAVHTRGLDFACDIYIWAPLAGTCGVLLLSLVITLYCKRGRKKLLYIFKQPFMRPVQTT |
| | QEEDGCSCRFPEEEEGGCELRVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLSTATKDTYDALHMQALPPR |
| 96 | DIQMTQTTSSLSASLGDRVTISCRASQDISKYLNWYQQKPDGTVKLLIYHTSRLHSGVPSRFSGSGS |
| | GTDYSLTISNLEQEDIATYFCQQGNTLPYTFGGGTKLEITGGGGSGGGGSGGGGSEVKLQESGPGLV |
| | APSQSLSVTCTVSGVSLPDYGVSWIRQPPRKGLEWLGVIWGSETTYYNSALKSRLTIIKDNSKSQVF |
| | LKMNSLQTDDTAIYYCAKHYYYGGSYAMDYWGQGTSVTVSSTTTPAPRPPTPAPTIASQPLSLRPEA |
| | CRPAAGGAVHTRGLDFACDIYIWAPLAGTCGVLLLSLVITLYCKRGRKKLLYIFKQPFMRPVQTTQE |
| | EDGCSCRFPEEEEGGCELRVKFSRSADAPAYKQGQNQLYNELNLGRREEYDVLDKRRGRDPEMGGKP |
| | RRKNPQEGLYNELQKDKMAEAYSEIGMKGERRRGKGHDGLYQGLSTATKDTYDALHMQALPPR |
| 97 | DIQMTQTTSSLSASLGDRVTISCRASQDISKYLNWYQQKPDGTVKLLIYHTSRLHSGVPSRFSGSGS |
| | GTDYSLTISNLEQEDIATYFCQQGNTLPYTFGGGTKLEITGGGGSGGGGSGGGGSEVKLQESGPGLV |
| | APSQSLSVTCTVSGVSLPDYGVSWIRQPPRKGLEWLGVIWGSETTYYNSALKSRLTIIKDNSKSQVF |
| | LKMNSLQTDDTAIYYCAKHYYYGGSYAMDYWGQGTSVTVSSKIEVMYPPPYLDNEKSNGTIIHVKGK |
| | HLCPSPLFPGPSKPFWVLVVVGGVLACYSLLVTVAFIIFWVRSKRSRLLHSDYMNMTPRRPGPTRKH |
| | YQPYAPPRDFAAYRSRVKFSRSADAPAYQQGQNQLYNELNLGRREEYDVLDKRRGRDPEMGGKPRRK |
| | NPQEGLYNELQKDKMAEAYSEIGMKGERRRGKGHDGLYQGLSTATKDTYDALHMQALPPR |

### 3. Dimerization Activated Receptor Initiation Complexes

In particular embodiments, a retroviral vector comprises a polynucleotide encoding a promoter operably linked to a polynucleotide encoding a DARIC. A DARIC provides a binding component and a signaling component that are each expressed as separate fusion proteins comprising an extracellular multimerization domain. The multimerization domains of the components associate by binding to a bridging factor (see U.S. Pat. Appl. No. 2016/0311901, which is incorporated herein by reference in its entirety). Although the bridging factor leads to formation of the DARIC system components, formation of the DARIC complex does not produce significant signaling on its own. The described DARIC complexes contemplated herein only initiate physiologically relevant signals when the DARIC binding component binds to a target antigen expressed or displayed on a target cell. In particular embodiments, a DARIC binding component comprises an antigen binding domain that binds to an antigen selected from the group consisting of: FRα, avβ6 integrin, BCMA, CD276, B7-H6, CAIX, CD16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGFR, EGFRvIII, EGP2, EGP40, EPCAM, EPHA2, FAP, FCRL5, AchR, GD2, GD3, GPC3, GPCR5D, HER2, IL-11Rα, IL-13Rα2, LAGE-1A, Lambda, LeY, L1-CAM, MAGE-A1, MAGE-A3, MAGE-A4, MAGE-A6, MAGEA10, MelanA or MART1, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

### F. Retroviral Vectors

Recombinant retroviruses contemplated herein comprise a retroviral vector. The retroviral vector is based on a retrovirus genome that has been engineered to remove viral accessory proteins but leave elements intact for packaging, reverse transcription and integration. In particular embodiments, a recombinant virus contains two copies of a vector, a genomic RNA comprising backbone sequences derived from one or more retroviruses selected from the group consisting of an alpharetrovirus, a betaretrovirus, a gammaretrovirus, a deltaretrovirus, an epsilonretrovirus, or a spumavirus.

In particular embodiments, a retroviral vector comprises, a 5′ LTR, a Psi (Ψ) packaging sequence, a polynucleotide encoding a promoter operably linked to a polynucleotide encoding an engineered antigen receptor and optionally a woodchuck hepatitis virus post-transcriptional regulatory element (WPRE), and a 3′ LTR. The polynucleotide encoding the engineered antigen receptor can be expressed from a retroviral LTR promoter or an internal promoter.

In particular embodiments, a retroviral vector comprises, a 5′ LTR, a Psi (Ψ) packaging sequence, a polynucleotide encoding a promoter operably linked to a polynucleotide encoding an engineered antigen receptor and optionally a woodchuck hepatitis virus post-transcriptional regulatory element (WPRE), a 3′ LTR, and a polyA tail.

In particular embodiments, a retroviral vector comprises, an R element and a U5 element, a Psi (Ψ) packaging sequence, a polynucleotide encoding a promoter operably linked to a polynucleotide encoding an engineered antigen receptor and optionally a woodchuck hepatitis virus post-transcriptional regulatory element (WPRE), a U3 element and an R element, a polyadenylation signal and a polyA tail.

In particular embodiments, a retroviral vector, e.g., a SIN retroviral vector, comprises, from 5′ to 3′: an R element and a U5 element; a Psi (Ψ) packaging sequence; a polynucleotide encoding a promoter selected from the group consisting of an EF1α promoter, a cytomegalovirus (CMV) promoter, a Moloney murine leukemia virus (MoMLV) promoter, a Rous sarcoma virus (RSV) promoter, a 3-phosphoglycerate kinase (PGK-1) promoter, a herpes simplex virus (HSV) (thymidine kinase) promoter, a chicken β-actin (CAG) promoter, an SV40 promoter, an SV40/CD43 promoter, and an MNDU3 promoter; a polynucleotide encoding an engineered antigen receptor, optionally a CAR that binds CD19, CD20, CD22, BMCA, CLL-1, CD33, CD79A, CD79B, mesothelin, or MUC16; optionally a WPRE, a U3 element and an R element, a polyadenylation signal and a polyA tail.

In particular embodiments, a retroviral vector is derived from one or more retroviral genomes of an alpharetrovirus, a betaretrovirus, a gammaretrovirus, a deltaretrovirus, an epsilonretrovirus, or a spumavirus.

In particular embodiments, a recombinant retrovirus comprises a polynucleotide sequence encoding a retroviral vector derived from a retrovirus selected from the group consisting of SMRV, BaEV, RD114, FeLV, GALV, MLV, MoMLV, PERV, REV, XMRV, and HFV.

In particular embodiments, a recombinant retrovirus comprises a polynucleotide sequence encoding a retroviral vector comprising U3, R and U5 elements, and optionally an LTR promoter, from one or more retroviruses selected from the group consisting of an alpharetrovirus, a betaretrovirus, a gammaretrovirus, a deltaretrovirus, an epsilonretrovirus, or a spumavirus.

In particular embodiments, a recombinant retrovirus comprises a polynucleotide sequence encoding a retroviral vector comprising U3, R and U5 elements, and optionally an LTR promoter, from a retrovirus selected from the group consisting of SMRV, BaEV, RD114, FeLV, GALV, MLV, MoMLV, PERV, REV, XMRV, and HFV.

In particular embodiments, a retrovirus is manufactured using a multi-plasmid system comprising an envelope plasmid encoding a viral envelope glycoprotein (e.g., one or more mutated viral envelope glycoproteins that retain fusogenic activity and lack cognate receptor binding activity) and one or more tropism polypeptides, a packaging plasmid encoding gag-pol and a transfer plasmid or vector. The “transfer plasmid” or “transfer vector” encodes a viral genomic RNA (e.g., vector backbone) comprising the polynucleotide sequence that is delivered by recombinant retrovirus to a target cell. In particular embodiments, a transfer plasmid comprises one or more transgene sequences of interest flanked by long terminal repeat (LTR) sequences, which facilitate packaging, reverse transcription and integration of the viral vector and associated polynucleotide sequences into the host genome. Viral vectors contemplated herein are replication incompetent, i.e., lack the genetic elements necessary for generation of infective particles in the host cell. For example, the viral vector may be designed with a deletion of the 3′ LTR, rendering the virus “self-inactivating” (SIN).

In particular embodiments, a transfer plasmid comprises a promoter selected from the group consisting of an RSV promoter, an SV40 promoter, a CMV promoter, an MoMLV promoter, an RSV promoter, and an HSV-TK promoter operably linked to a polynucleotide sequence encoding a retroviral vector comprising: an R element and a U5 element; a Psi (Ψ) packaging sequence; a polynucleotide encoding an engineered antigen receptor, optionally a CAR that binds CD19, CD20, CD22, BMCA, CLL-1, CD33, CD38, CD79A, CD79B, GPCR5D, mesothelin, or MUC16; optionally a WPRE, a U3 element and an R element and a polyadenylation signal.

In particular embodiments, a recombinant retrovirus comprises one or more components of a gene editing payload including but not limited to polynucleotides intended for insertion into the genome of the target cell and/or gene editing systems (CRISPR-Cas, meganucleases, homing endonucleases, zinc finger enzymes and the like).

### G. IMMUNE EFFECTOR CELLS

In particular embodiments, a recombinant retrovirus contemplated herein is engineered to transduce one or more immune effector cells. Non-limiting examples of immune effector cells that can be transduced with the recombinant retrovirus contemplated herein include but are not limited to T lymphocytes, B lymphocytes, dendritic cells (DC), Treg cells, natural killer (NK) cells, natural killer T (NKT) cells, and macrophages.

T lymphocytes (“T cells”) are a type of lymphocyte (itself a type of white blood cell) that play a central role in cell-mediated immunity. Primary T cells include but are not limited to naïve T cells, MHC-restricted T cells, CD3⁺ T cells, CD4⁺ T cells, CD8⁺ T cells, or tumor infiltrating lymphocytes (TILs). T cells may be autologous or allogeneic.

Natural killer (NK) cells are cytotoxic lymphocytes that constitute a major component of the innate immune system. NK cells do not express T cell antigen receptors (TCR), CD3 or surface immunoglobulins (Ig) B cell receptor, but usually express the surface markers CD 16 (FcyRIII) and CD56 in humans.

### H. Polypeptides

Various polypeptides are contemplated herein, including, but not limited to, engineered antigen receptors and components thereof, mutated viral envelope glycoproteins, non-viral membrane bound tropism polypeptides, chimeric antigen receptors, and the like. In particular embodiments, a polypeptide comprises an amino acid sequence set forth in any one of SEQ ID NOs: 1-111. “Polypeptide,” “peptide” and “protein” are used interchangeably, unless specified to the contrary, and according to conventional meaning, i.e., as a sequence of amino acids. Polypeptides can be prepared using recombinant and/or synthetic techniques. Polypeptides are not limited to a specific length, e.g., they may comprise a full-length protein sequence, a fragment of a full-length protein, or a fusion protein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more substitutions, deletions, additions and/or insertions. In particular embodiments, a polypeptide variant comprises at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to a reference sequence contemplated herein, typically where the variant maintains at least one biological activity of the reference sequence.

Polypeptide variants include “polypeptide fragments.” A polypeptide fragment is a polypeptide that is not full length and comprises one or more terminal truncations and/or internal deletions.

In certain embodiments, a polypeptide variant comprises one or more conservative substitutions or disruptive substitutions. A “conservative substitution” is one in which an amino acid is substituted for another amino acid that has similar properties. In particular embodiments, polypeptide variants contemplated herein comprise one or more conservative amino acid changes compared to a reference polypeptide. In particular embodiments, a conservative amino acid substitution involves substituting an amino acid with an amino acid having a related side chain. A “disruptive substitution” is one in which an amino acid is substituted for another amino acid that has different properties, e.g., polar vs. non-polar, bulky vs. non-bulky, charged vs. uncharged, acidic vs. basic. In particular embodiments, polypeptide variants contemplated herein comprise one or more disruptive amino acid changes compared to a reference polypeptide. In particular embodiments, a disruptive amino acid substitution involves substituting an amino acid with an amino acid having an unrelated side chain or side change with a different chemical property. Guidance in determining which amino acid residues can be substituted, inserted, or deleted can be found using computer programs well known in the art, such as DNASTAR, DNA Strider, Geneious, Mac Vector, or Vector NTI software.

Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In particular embodiments, a conservative amino acid substitution refers to substituting amino acids within the same group or family.

Those of skill in this art recognize that, in general, conservative single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224), whereas disruptive single amino acid substitutions may.

In particular embodiments, a conservative amino acid substitution refers to substituting amino acids having a similar hydropathic index or score. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In particular embodiments, a conservative amino acid substitution refers to substituting amino acids having a similar hydropathic index or score. In particular embodiments, substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

In particular embodiments, a conservative amino acid substitution refers to substituting amino acids having a similar hydrophilic index or score. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In particular embodiments, a conservative amino acid substitution refers to substituting amino acids having a similar hydrophilic index or score. In particular embodiments, substitution of amino acids whose hydrophilic indices are substitution of amino acids whose hydrophilicity values are within ±2 is preferred, those within ±1 are particularly preferred, and those within ±0.5 are even more particularly preferred.

In particular embodiments, a conservative amino acid substitution may be based on the relative similarity of the amino acid side-chain substituents, for example, their hydrophobicity, hydrophilicity, charge, size, and the like. In particular embodiments, a disruptive amino acid substitution may be based on the relative dissimilarity of the amino acid side-chain substituents, for example, their hydrophobicity, hydrophilicity, charge, size, and the like.

In particular embodiments, a polypeptide comprises a vesiculovirus envelope glycoprotein G comprising one or more amino acid substitutions at positions 47 and/or 354 of the mature polypeptide lacking a signal peptide. In particular embodiments, a polypeptide comprises a mutated cocal virus envelope glycoprotein (COCV-G) or a mutated vesicular stomatitis Indiana virus envelope glycoprotein (VSIV-G), wherein the COCV-G or VSIV-G comprises amino acid substitutions at positions 47 and 354.

In particular embodiments, a retroviral vector is designed or constructed to express two or more polypeptides. Polynucleotide sequences encoding two or more polypeptides can be separated by an IRES sequence or a polynucleotide sequence encoding a ribosomal skip sequence as disclosed elsewhere herein.

Fusion polypeptides can comprise one or more polypeptide domains or segments including, but are not limited to signal peptides, antibodies and antigen binding domains, co-stimulatory domains, primary signaling domains, polypeptide linkers, and polypeptide cleavage signals.

Fusion polypeptides may optionally comprise one or more polypeptide linkers that can be used to link the one or more polypeptides or domains within a polypeptide.

Exemplary linkers include, but are not limited to the following amino acid sequences: glycine polymers; glycine-serine polymers; glycine-alanine polymers; alanine-serine polymers; GGG; DGGGS (SEQ ID NO: 98); TGEKP (SEQ ID NO: 99); GGRR (SEQ ID NO: 100); GGGGS (SEQ ID NO: 101); GGGGSGGGGS (SEQ ID NO: 102); GGGGSGGGGSGGGGS (SEQ ID NO: 103); GGGGSGGGGSGGGGSGGGGS (SEQ ID NO: 104); GGGGSGGGGSGGGGSGGGGSGGGGS (SEQ ID NO: 105); EGKSSGSGSESKVD (SEQ ID NO: 106); KESGSVSSEQLAQFRSLD (SEQ ID NO: 107); GGRRGGGS (SEQ ID NO: 108); LRQRDGERP (SEQ ID NO: 109); LRQKDGGGSERP (SEQ ID NO: 110); and LRQKDGGGSGGGSERP (SEQ ID NO: 111).

Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

In particular embodiments, the polypeptide cleavage signal is a viral self-cleaving peptide or ribosomal skipping sequence.

Illustrative examples of ribosomal skipping sequences include, but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide. In particular embodiments, a viral 2A peptide is selected from the group consisting of: a foot-and-mouth disease virus (FMDV) 2A peptide, an equine rhinitis A vims (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

### I. Polynucleotides

Various polynucleotides are contemplated herein including, but not limited to, a transfer vectors, retroviral vectors, packaging plasmids, and polynucleotide encoding engineered antigen receptors and components thereof, mutated viral envelope glycoproteins, non-viral membrane bound tropism polypeptides. In particular embodiments, a polynucleotide encodes a polypeptide comprising an amino acid sequence set forth in any one of SEQ ID NOs: 1-111. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, synthetic RNA, synthetic mRNA, genomic RNA (viral genomic RNA), genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc, 151, 152, 153, etc:, 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state. In particular embodiments, an isolated polynucleotide is a synthetic polynucleotide, a semi-synthetic polynucleotide, or a polynucleotide obtained or derived from a recombinant source, or other polynucleotide that does not exist in nature and that has been made by the hand of man.

In various embodiments, a polynucleotide comprises a genomic RNA encoding a retroviral vector.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Included are polynucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein.

“Expression control sequences,” “control elements,” or “regulatory sequences” present in a vector are those non-translated regions of the vector including but not limited to an origin of replication, selection cassettes, promoters, enhancers, translation initiation signals (Shine Dalgamo sequence or Kozak sequence) introns, a polyadenylation sequence, 5′ and 3′ untranslated regions, all of which interact with host cellular proteins to carry out transcription and translation.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

Illustrative expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), P-kinesin (P-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken P-actin (CAG) promoter, a P-actin promoter and a myeloproliferative sarcoma virus enhancer, negative control region deleted, dl587rev primer-binding site substituted (MND) U3 promoter (Haas et al., Journal of Virology. 2003; 77(17): 9439-9450).

Elements directing the efficient termination and polyadenylation of heterologous nucleic acid transcripts increases heterologous gene expression. Transcription termination signals are generally found downstream of the polyadenylation signal. In particular embodiments, vectors comprise a polyadenylation sequence 3′ to a sequence to be transcribed and/or expressed. The terms “polyadenylation (or polyA) site,” “polyadenylation (or polyA) signal” or “polyadenylation (or polyA) sequence” as used herein denotes a DNA sequence which directs both the termination and polyadenylation of the nascent RNA transcript by RNA polymerase II. Polyadenylation signals can promote mRNA stability by addition of a polyA tail to the 3′ end of the coding sequence and thus, contribute to increased translational efficiency. Cleavage and polyadenylation are directed by a poly(A) signal in the RNA. The core poly(A) signal for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) signal is an ideal polyA signal (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art. In particular embodiments, the poly(A) sequence is synthetic.

In particular embodiments, retroviral vectors comprising polynucleotides contemplated herein are administered directly to an organism for transduction of cells in vivo. In particular embodiments, retroviral vectors are delivered in vivo by administration to an individual patient, typically by systemic administration (e.g., intravenous, intraperitoneal, intramuscular, subdermal, or intracranial infusion) or topical application, as described below. In particular embodiments, retroviral vectors comprising polynucleotides contemplated herein are administered to cells for transduction ex vivo.

### J. Compositions and Formulations

Formulations and compositions contemplated herein comprise a recombinant retrovirus formulated in pharmaceutically acceptable or physiologically-acceptable compositions for administration to a cell, tissue, organ, or an animal, either alone, or in combination with one or more other modalities of therapy.

“Pharmaceutically acceptable” refers to molecular entities and compositions that do not produce excessive toxicity, irritation, allergic response, or other problem or complication, commensurate with a reasonable benefit/risk ratio when administered to a human.

The term “carrier” includes any and all solvents, dispersion media, vehicles, coatings, diluents, antibacterial and antifungal agents, isotonic and absorption delaying agents, buffers, carrier solutions, suspensions, colloids, and the like.

A “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion.

The pharmaceutical compositions that contain a recombinant retrovirus comprising a vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The retrovirus can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the particles contemplated herein.

In particular embodiments, a recombinant retrovirus (e.g., lentivirus), or a pharmaceutical composition comprising that recombinant retrovirus, is effective when administered systemically (e.g., intravenously). In certain embodiments, the retroviral vectors induce expression of an engineered antigen receptor in transduced immune cells when a recombinant retrovirus is administered systemically.

In various embodiments, the pharmaceutical compositions contain vehicles (e.g., carriers, diluents and excipients) that are pharmaceutically acceptable for a formulation capable of being injected.

In particular embodiments, compositions are formulated for in vivo administration.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation. In certain embodiments, compositions contemplated herein are administered parenterally, intravenously, intramuscularly, or intraperitoneally.

A recombinant retrovirus may be used to transduce cells in vivo at an any effective dosage. In particular embodiments, a recombinant retrovirus is administered to a subject in vivo.

It would be understood by the skilled artisan that particular embodiments contemplated herein may comprise other formulations, such as those that are well known in the pharmaceutical art, and are described, for example, in Remington: The Science and Practice of Pharmacy, Volume I and Volume II. 23^{rd} Edition. Edited by Adeboye Adejare. Academic Press, 2020, which is incorporated by reference herein, in its entirety.

### K. Method of Treatment

In various embodiments, a recombinant retrovirus and compositions contemplated herein are used to treat, prevent, or ameliorate at least one symptom of a disease, disorder, or condition. In particular embodiments, the disease, disorder, or condition is cancer. In particular embodiments, the cancer is a hematological malignancy or a solid tumor. In particular embodiments, the subject has relapsed or is refractory to treatment with a prior anti-cancer therapeutic.

In some embodiments, the cancer is a hematological malignancy.

In particular embodiments, a recombinant retrovirus contemplated herein treats, prevents, or ameliorates at least one symptom of a multiple myeloma, a smoldering multiple myeloma, a monoclonal gammopathy of undetermined significance (MGUS), an acute lymphoblastic leukemia (ALL), a diffuse large B cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B cell lymphoblastic leukemia, a precursor B cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B cell lymphoma (MZL), a mucosa-associated lymphatic tissue lymphoma (MALT), plasma cell leukemia, anaplastic large-cell lymphoma (ALCL), leukemia or lymphoma.

In particular embodiments, a recombinant retrovirus contemplated herein treats, prevents, or ameliorates at least one symptom of a CD19-expressing B cell malignancy.

In particular embodiments, a recombinant retrovirus contemplated herein treats, prevents, or ameliorates at least one symptom of a BCMA-expressing B cell malignancy.

In particular embodiments, a recombinant retrovirus contemplated herein treats, prevents, or ameliorates at least one symptom of a solid tumor.

In particular embodiments, a recombinant retrovirus contemplated herein treats, prevents, or ameliorates at least one symptom of prostate cancer, lung cancer, non-small cell lung cancer (NSCLC), liver cancer, cervical cancer, colon cancer, breast cancer, ovarian cancer, endometrial cancer, pancreatic cancer, melanoma, esophageal cancer, gastric cancer, stomach cancer, renal carcinoma, bladder cancer, hepatocellular carcinoma, renal cell carcinoma, urothelial carcinoma, head and neck cancer, glioma, glioblastoma, colorectal cancer, thyroid cancer, epithelial cancers, or adenocarcinomas.

In particular embodiments, a recombinant retrovirus is administered in combination with one or more anti-cancer therapies including, but not limited to, an autologous stem cell transplant (ASCT), radiation, surgery, a chemotherapeutic agent, an immunomodulatory agent and a targeted cancer therapy.

In particular embodiments, the one or more anti-cancer therapies is selected from the group consisting of 6-mercaptopurine, abiraterone, alemtuzumab, all-trans retinoic acid, anastrozole, aprepitant, arsenic trioxide, atezolizumab, azacytidine, bafetinib, bevacizumab, bleomycin, bortezomib, bosutinib, cabazitaxel, capecitabine, carboplatin, carfilzomib, cetuximab, cisplatin, cladribine, corticosteroid, crizotinib, cyclophosphamide, cytarabine, dasatinib, daunorubicin, danusertib, decitabine, denosumab, dexamethasone, docetaxel, doxorubicin, elotozumab, eribulin, erlotinib, etoposide, everolimus, exemestane, filgrastim, fludarabine, fluorouracil, fulvestrant, gemcitabine, hydroxyurea, idarubicin, imatinib, imiquimod, ipilimumab, ixabepilone, ixazomib, lapatinib, lenalidomide, letrozole, leuprolide, melphalan, methotrexate, mitoxantrone, nilotinib, nivolumab, oxaliplatin, paclitaxel, palonosetron, pembrolizumab, pemetrexed, pomalidomide, ponatinib, prednisone, radium-223, rituximab, saracatinib, sipuleucel-T, sorafenib, sunitinib, tamoxifen, temozolomide, temsirolimus, thalidomide, tinorelbine, topotecan, tozasertib, trastuzumab, vincristine, and zoledronic acid.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. Those of skill in the art will readily recognize a variety of noncritical parameters that could be changed or modified to yield essentially similar results.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A recombinant retrovirus comprising:
(a) a viral envelope comprising (i) one or more mutated viral envelope glycoproteins that retain fusogenic activity and lack cognate receptor binding activity and (ii) a non-viral membrane-bound tropism polypeptide, and
(b) a recombinant retroviral vector comprising a polynucleotide encoding a promoter operably linked to a polynucleotide encoding one or more engineered antigen receptors;
wherein the retroviral vector is engineered or derived from a retrovirus selected from the group consisting of: an alpharetrovirus genome, a betaretrovirus genome, a gammaretrovirus genome, a deltaretrovirus genome, an epsilonretrovirus genome, a simiispumavirus genome, a bovispumavirus genome, an equispumavirus genome, a felispumavirus genome, and a prosimiispumavirus genome.

2. The recombinant retrovirus of claim 1, wherein the retrovirus comprises a gag protein and a pol protein of an alpharetrovirus, a betaretrovirus, a gammaretrovirus, a deltaretrovirus, an epsilonretrovirus, a simiispumavirus, a bovispumavirus, an equispumavirus, a felispumavirus, or a prosimiispumavirus; and a 5′ LTR and a 3′ LTR isolated, obtained, or derived from a retrovirus genome selected from the group consisting of: an alpharetrovirus genome, a betaretrovirus genome, a gammaretrovirus genome, a deltaretrovirus genome, an epsilonretrovirus genome, a simiispumavirus genome, a bovispumavirus genome, an equispumavirus genome, a felispumavirus genome, and a prosimiispumavirus genome.

3. The recombinant retrovirus of claim 1 or claim 2, wherein the one or more mutated viral envelope glycoproteins comprise a vesiculovirus envelope glycoprotein, one or more morbillivirus envelope glycoproteins or one or more henipavirus envelope glycoproteins.

4. The recombinant retrovirus of claim 3, wherein the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

5. The recombinant retrovirus of claim 3 or claim 4, wherein the vesiculovirus envelope glycoprotein is a vesiculovirus G protein.

6. The recombinant retrovirus of any one of claims 3 to 5, wherein the vesiculovirus G protein is a COCV G glycoprotein (COCV-G) or a VSIV G glycoprotein (VSIV-G).

7. The recombinant retrovirus of claim 6, wherein the VSIV-G envelope protein comprises one or more of:
(a) one or more amino acid substitutions at H8, N9, Q10, K47, K50, A51, S183, S179, N180, I182, M184, Y209, T214, I347, T350, T352, E353, and R354;
(b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209;
(c) amino acid substitutions at K47 and R354; or
(d) a deletion of residues 1-8.

8. The recombinant retrovirus of claim 6 or claim 7, wherein the VSIV-G envelope protein comprises one or more amino acid substitutions at H8, K47, Y209, and R354.

9. The recombinant retrovirus of any one of claims 6 to 8, wherein the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

10. The recombinant retrovirus of any one of claims 6 to 9, wherein the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

11. The recombinant retrovirus of any one of claims 3 to 6, wherein the vesiculovirus G protein is COCV-G.

12. The recombinant retrovirus of claim 11, wherein the COCV-G envelope protein comprises one or more amino acid substitutions at K47 and R354.

13. The recombinant retrovirus of claim 11 or claim 12, wherein the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

14. The recombinant retrovirus of any one of claims 11 to 13, wherein the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=Q and X₂=Q.

15. The recombinant retrovirus of claim 3, wherein the one or more morbillivirus envelope glycoproteins are measles virus F (MV-F) and measles virus H (MV-H).

16. The recombinant retrovirus of claim 15, wherein the MV-H protein comprises one or more amino acid substitutions at Y481, R533, S548, and F549.

17. The recombinant retrovirus of claim 15 or claim 16, wherein the MV-H protein comprises one or more amino acid substitutions selected from the group consisting of: Y481A, R533A, S548L, and F549S.

18. The recombinant retrovirus of claim 3, wherein the one or more henipavirus envelope glycoproteins are nipah virus F (NiV-F) and nipah virus G (NiV-G).

19. The recombinant retrovirus of claim 18, wherein the NiV-G protein comprises one or more amino acid substitutions at E501, W504, Q530, and E533.

20. The recombinant retrovirus of claim 18 or claim 19, wherein the NiV-G protein comprises one or more amino acid substitutions at E501A, W504A, Q530A, and E533A.

21. The recombinant retrovirus of any one of claims 1 to 20, wherein the non-viral membrane-bound tropism polypeptide comprises an extracellular antigen targeting domain, a spacer domain, and a transmembrane domain.

22. The recombinant retrovirus of claim 21, wherein the extracellular antigen targeting domain binds an antigen expressed on an immune effector cell.

23. The recombinant retrovirus of claim 21 or claim 22, wherein the extracellular antigen targeting domain binds an antigen expressed on an immune effector cell selected from the group consisting of: the alpha, beta, gamma, or delta chain of the T cell receptor, CD2, CD3δ, CD3ε CD3γ, CD4, CD5, CD7, CD8α, and CD8β.

24. The recombinant retrovirus of any one of claims 21 to 23, wherein the extracellular antigen targeting domain comprises an antibody or antigen binding fragment thereof that binds an antigen expressed on the immune effector cell.

25. The recombinant retrovirus of any one of claims 21 to 24, wherein the extracellular antigen targeting domain comprises an anti-CD3 antibody or antigen binding fragment selected from the group consisting of OKT3, UCHT1, YTH12.5, TR66, and variants thereof, e.g., teplizumab, and antibodies and antigen binding fragments that have at least 80%, at least 85%, at least 90%, at least 95%, at least 99%, or 100% identity thereto.

26. The recombinant retrovirus of any one of claims 1 to 25, wherein the viral envelope further comprises one or more secondary tropism polypeptides.

27. The recombinant retrovirus of claim 26, wherein the one or more secondary tropism polypeptides comprise one or more of CD80, CD86, CD137L, OX40L, and ICOSL.

28. The recombinant retrovirus of any one of claims 1 to 27, wherein the promoter is an EF1α promoter, a cytomegalovirus (CMV) promoter, a CAG promoter, an SV40 promoter, an SV40/CD43 promoter, a murine stem cell virus (MSCV) promoter, a phosphoglycerate kinase (PGK) promoter, or an MNDU3 promoter.

29. The recombinant retrovirus of any one of claims 1 to 28, wherein the engineered antigen receptor is a chimeric antigen receptor (CAR), a T cell receptor (TCR), or a dimerization activated receptor initiation complex (DARIC).

30. The recombinant retrovirus of any one or claims 1 to 29, wherein the engineered antigen receptor comprises an extracellular antigen binding domain that binds to alpha folate receptor (FRα), αvβ6 integrin, B cell maturation antigen (BCMA), B7-H3 (CD276), B7-H6, carbonic anhydrase IX (CAIX), CD16, CD 19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant IP (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), G Protein-Coupled Receptor Class C Group 5 Member D (GPCR5D), EGFR family including ErbB2 (HER2), IL-10Rα, IL-13Rα2, Kappa, cancer/testis antigen 2 (LAGE-1A), Lambda, Lewis-Y (LeY), LI cell adhesion molecule (L1-CAM), melanoma antigen gene (MAGE)-A1, MAGE-A3, MAGE-A4, MAGE-A6, MAGE A10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL 16-binding protein (ULBP) 1, ULBP2, ULBP3, ULBP4, ULBP5, ULBP6, vascular endothelial growth factor receptor 2 (VEGFR2), or Wilms tumor 1 (WT-1)

31. The recombinant retrovirus of any one or claims 1 to 30, wherein the engineered antigen receptor is a CAR.

32. The recombinant retrovirus of claim 31, wherein the CAR comprises an extracellular antigen binding domain comprising an antibody or antigen binding fragment thereof.

33. The recombinant retrovirus of claim 32, wherein the antibody or antigen binding fragment thereof is selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), a single-domain antibody (sdAb, a camelid VHH, Nanobody) and a centyrin.

34. The recombinant retrovirus of claim 32 or claim 33, wherein the antibody or antigen binding fragment thereof comprises one or more scFv and/or one or more VHHs.

35. The recombinant retrovirus of any one of claims 31 to 34, wherein the CAR comprises a hinge domain, optionally a hinge domain selected from the group consisting of an IgG4 hinge, a CD8α hinge, and a CD28 hinge.

36. The recombinant retrovirus of any one of claims 31 to 35, wherein the CAR comprises a transmembrane domain isolated, obtained, or derived from a polypeptide selected from the group consisting of the alpha, beta, gamma, or delta chain of the T cell receptor, CD2, CD3, CD4, CD8α, and CD28.

37. The recombinant retrovirus of any one of claims 31 to 36, wherein the CAR comprises one or more intracellular primary signaling domains isolated, obtained, or derived from a polypeptide selected from the group consisting of: FcRγ, FcRβ, CD3ζ, CD79a, CD79b, and CD66d.

38. The recombinant retrovirus of any one of claims 31 to 37, wherein the CAR comprises one or more intracellular costimulatory signaling domains isolated, obtained, or derived from a polypeptide selected from the group consisting of: CD2, CD7, CD11a, CD27, CD28, CD30, CD40, CD134 (OX40), CD137 (4-1BB), CD278 (ICOS), and TNF receptor superfamily member 14 (TNFRS14).

39. The recombinant retrovirus of any one of claims 31 to 38, wherein the CAR comprises an amino acid sequence set forth in any one of SEQ ID NOs: 93 to 97.

40. The recombinant retrovirus of any one of claims 31 to 39, wherein the retroviral vector further comprises one or more polynucleotides encoding a P2A self-cleaving peptide, a T2A self-cleaving peptide, or an E2A self-cleaving peptide.

41. The recombinant retrovirus of claim 40, wherein the retroviral vector further comprises a polynucleotide encoding a prodrug activator enzyme.

42. The recombinant retrovirus of claim 41, wherein the prodrug activator enzyme is cytosine deaminase or thymidine kinase.

43. A method of manufacturing the recombinant retrovirus of any one of claims 1 to 42 comprising transfecting a packaging cell line that expresses the one or more mutated viral envelope glycoproteins that retain fusogenic activity and lack cognate receptor binding activity, the non-viral membrane-bound tropism polypeptide, optionally the secondary tropism polypeptide, and the gag and pol of claim 2, with a plasmid encoding the retroviral vector; and culturing the packaging cell to manufacture the recombinant retrovirus.

44. A method of manufacturing the recombinant retrovirus of any one of claims 1 to 42 comprising transfecting a packaging cell line that expresses the gag and pol of claim 2 with one or more plasmids encoding the one or more mutated viral envelope glycoproteins that retain fusogenic activity and lack cognate receptor binding activity, the non-viral membrane-bound tropism polypeptide, and optionally the secondary tropism polypeptide; and culturing the packaging cell to manufacture the recombinant retrovirus.

45. A composition comprising the recombinant retrovirus of any one of claims 1 to 42.

46. A pharmaceutical composition comprising the recombinant retrovirus of any one of claims 1 to 42 and a pharmaceutically acceptable carrier.

47. A method of treating a subject in need thereof comprising administering to the subject an effective amount of the recombinant retrovirus of any one of claims 1 to 42.

48. The method of claim 47, wherein the amount is effective to transduce an immune effector cell in the subject.


SEQUENCE LISTING (111 sequences)

SEQ ID NO: 1 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 495 aa, synthetic construct
  VARIANT 41: note = I,L
  VARIANT 47: note = A,Q
  VARIANT 80: note = H,Q
  VARIANT 354: note = A,Q
KFTIVFPHNQKGNWKNVPSNYHYCPSSSDLNWHNDLIGTAXQVKMPXSHKAIQADGWMCHASKWVTTCDFRWYGPKYITXSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEXELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 4 - protein, 495 aa, synthetic construct
  VARIANT 47: note = A,Q
  VARIANT 354: note = A,Q
KFSIVFPQSQKGNWKNVPSSYHYCPSSSDQNWHNDLLGITMKVKMPXTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEXELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - protein, 497 aa, synthetic construct
QIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 6 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTN

SEQ ID NO: 7 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTN

SEQ ID NO: 8 - protein, 497 aa, synthetic construct
LHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 9 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 10 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 11 - protein, 243 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSS

SEQ ID NO: 12 - protein, 240 aa, synthetic construct
DIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSS

SEQ ID NO: 13 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 15 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 21 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 23 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 25 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 26 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 27 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 29 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 30 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 31 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 32 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 33 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 34 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 35 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 36 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 37 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 38 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 39 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 495 aa, synthetic construct; identical to SEQ ID NO: 40

SEQ ID NO: 45 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 47 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 48 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 50 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 51 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 52 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 53 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 54 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 55 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 56 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 57 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 58 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 59 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 60 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 61 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 62 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 63 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 64 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 75 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 76 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 77 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 78 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 79 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 80 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 81 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 82 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 83 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 84 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 85 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 86 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 87 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 88 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 89 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 90 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 91 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 92 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 93 - protein, 472 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 438 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 467 aa, synthetic construct
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 465 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 462 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 5 aa, synthetic construct
DGGGS

SEQ ID NO: 99 - protein, 5 aa, synthetic construct
TGEKP

SEQ ID NO: 100 - protein, 4 aa, synthetic construct
GGRR

SEQ ID NO: 101 - protein, 5 aa, synthetic construct
GGGGS

SEQ ID NO: 102 - protein, 10 aa, synthetic construct
GGGGSGGGGS

SEQ ID NO: 103 - protein, 15 aa, synthetic construct
GGGGSGGGGSGGGGS

SEQ ID NO: 104 - protein, 20 aa, synthetic construct
GGGGSGGGGSGGGGSGGGGS

SEQ ID NO: 105 - protein, 25 aa, synthetic construct
GGGGSGGGGSGGGGSGGGGSGGGGS

SEQ ID NO: 106 - protein, 14 aa, synthetic construct
EGKSSGSGSESKVD

SEQ ID NO: 107 - protein, 18 aa, synthetic construct
KESGSVSSEQLAQFRSLD

SEQ ID NO: 108 - protein, 8 aa, synthetic construct
GGRRGGGS

SEQ ID NO: 109 - protein, 9 aa, synthetic construct
LRQRDGERP

SEQ ID NO: 110 - protein, 12 aa, synthetic construct
LRQKDGGGSERP

SEQ ID NO: 111 - protein, 16 aa, synthetic construct
LRQKDGGGSGGGSERP